(19) United States
(12) Patent Application Publication
(10) Pub. No.: US 2012/0021437 A1
(43) Pub. Date: Jan. 26, 2012

(54) TETRANOR-PGDM/PGJM SPECIFIC IMMUNOGENS, ANTIBODIES, TRACERS, ASSAY KITS AND METHODS FOR MAKING SAME

(75) Inventors: Jeffrey K. Johnson, Ann Arbor, MI (US); Kirk M. Maxey, Ann Arbor, MI (US); Daniel J. Tew, Tipton, MI (US); Andriy M. Kornilov, Ypslianti, MI (US)

(73) Assignee: Cayman Chemical Company, Incorporated, Ann Arbor, MI (US)

(21) Appl. No.: 13/160,486

(22) Filed: Jun. 14, 2011

Related U.S. Application Data
(60) Provisional application No. 61/354,500, filed on Jun. 14, 2010.

(51) Int. Cl. G01N 33/566; C07K 14/765; C07K 14/77; C07K 14/47; C07K 16/18; C07K 1/107; C12N 9/96; C07D 311/82; G01N 33/577; C07K 14/00; C07K 14/435

(52) U.S. Cl. 435/7.9; 530/406; 530/363; 530/367; 530/386; 530/395; 435/188; 549/394; 436/501; 530/388.2; 530/389.8

(57) ABSTRACT

Tetranor-PGDM/tetranor-PGJM-specific antibodies, immunogens used to generate them, the processes of their manufacture, and their uses in assay kits for detecting and quantifying tetranor-PGDM and tetranor-PGJM in biological fluids for determining biosynthesis of PGD₂ in a subject or patient.

### CROSS REFERENCE TO RELATED APPLICATIONS

The present application claims priority from U.S. Provisional Application Ser. No. 61/354,500, filed Jun. 14, 2010, which is herein incorporated by reference.

### FIELD OF THE INVENTION

The present invention generally relates to immunogens and to the antibodies they generate that specifically bind tetranor-prostaglandin D₂ metabolite (tetranor-PGDM) and tetranor-prostaglandin J₂ metabolite (tetranor-PGJM), the methods for their manufacture, and their uses in assay kits for quantification of tetranor-PGDM or tetranor-PGJM in biological fluids. The present invention also generally relates to the manufacture and use of tracers in the associated assay kits.

### BACKGROUND OF THE INVENTION

All references, including patents and patent applications, are hereby incorporated by reference in their entireties.

Prostaglandins are found in virtually all tissues and glands and are extremely potent mediators of a diverse group of physiological processes (Funk, C. D. Science, 2001, 294, 1871-1875). Prostaglandins can participate in a wide range of body functions, such as the contraction and relaxation of smooth muscle (Andersson, K. E., Forman, A. Acta Pharmacol. Toxicol., 1978, 43 (Suppl. 2), 90-95), the dilation and constriction of blood vessels (Abramovich, D. R., Page, K. R., Parkin, A. M. L. Br. J. Pharmac., 1984, 81, 19-21), control of blood pressure (Anderson, R. J., Berl, T., McDonald, K. M., Schrier, R. W. Kidney International, 1976, 10, 205-215), and modulation of inflammation and immunity (Hata, A. N., Breyer, R. M. Pharmacol. Ther., 2004, 103(2), 147-166). In general, prostaglandins and related compounds are transported out of the cells in which they are produced and affect other target cells in close proximity to their site of secretion, mainly by interacting with the target cell's membrane-bound prostaglandin receptors to modulate some target cell function through signal transduction pathways. Prostaglandins and closely-related lipid mediators may also play a signaling role in the cells in which they are synthesized.

Prostaglandin D₂ (PGD₂) biosynthesis first involves a two-step enzymatic conversion of the ubiquitous polyunsaturated fatty acid (PUFA) arachidonic acid (AA) by a cyclooxygenase (COX) to the endoperoxide prostaglandin intermediate and common prostanoid precursor prostaglandin H₂ (PGH₂). Subsequent isomerization of PGH₂ to PGD₂ is mediated by the catalytic action of either the glutathione-dependent hematopoietic prostaglandin D synthase (H-PGDS) or the glutathione-independent lipocalin-like prostaglandin D synthase (L-PGDS) (Vitamins and Hormones, 58, 2000, 89-120). Production of PGD₂ in both intact human mast cells and platelets is known (Roberts, L., Sweetman, B., Lewis, R., et al. N. Engl. J. Med., 303, 1980, 1400-1404; Oelz, 0., Oelz, R., Knapp, H., et al. Prostaglandins, 19, 1977, 225-234) and the importance of PGD₂ and its metabolites in the resolution of inflammation has been studied (Gilroy, D., Colville-Nash, P., Willis, D. et al. Nat. Med., 5, 1999, 698-701).

Immunological challenge results in the release of PGD₂, the primary allergic and inflammatory mediator, from inflammatory cells. The biological effects of PGD₂ are transduced by at least two G protein coupled receptors (GPCRs), designated D prostanoid (DP) receptors DP₁ and CRTH₂/DP₂ (Boie, Y., Sawyer, N. Slipetz, D., et al. J. Biol. Chem., 270, 1995, 18910-18916; Nagata, K., Hirai, H. Prostaglandins Leukot. Essent. Fatty Acids, 69, 2003, 169-177). These receptors play a central role in airway inflammation (Spik, I., Brenuchon, C., Angeli, V., et al. J. Immunol., 174, 2005, 3703-3708; Urade, Y., Hayaishi, O. Vitamin and Hormones, 58, 2000, 89-120), systemic mastocytosis (Roberts, L., Sweetman, B., Lewis, R., et al. N. Engl. J. Med., 303, 1980, 1400-1404), and resolution.

As is the general case for all primary prostaglandins, direct quantitative measurements of PGD₂ formation have been limited due to its rapid biosynthesis and metabolism. The more-stable PGD₂ metabolites therefore are potentially useful indicators of PGD₂ biosynthesis (Song, W., Wang, M., Ricciotti E. et al. J. Biol. Chem., 283, 2008, 1179-1188; Cheng, Y., Wang, M., Yu, Y., et al. J. Clin. Invest., 116, 2006, 1391-1399; McAdam, B., Catella-Lawson, F., Mardini, I. et al. Proc. Natl. Acad. Sci. USA, 96, 1999, 272-277; Ellis, C., Smigel, M., Oates, J. et al. J. Biol. Chem., 254, 1979, 4152-4163). Tetranor-PGDM, or 9α-hydroxy-11,15-dioxo-13,14-dihydro-2,3,4,5-tetranor-prostan-1,20-dioic acid, is a major metabolite of PGD₂ found in human and murine urine (Song, W., Wang, M., Ricciotti E. et al. J. Biol. Chem., 283, 2008, 1179-1188). In human urine, tetranor-PGDM is significantly more abundant than the PGD₂ metabolites 11β-prostaglandin F_{2α} (11β-PGF_{2α}), or 9α,11β,15S-trihydroxy-prosta-5Z,13E-dien-1-oic acid, and 2,3-dinor-11β-PGF_{2α}, or 9α,11β,15S-trihydroxy-2,3-dinor-prosta-5Z,13E-dien-1-oic acid, and is the only endogenous PGD₂ metabolite detectable in murine urine by LC-MS. Normal levels of tetranor-PGDM in human and murine urine are 1.5 ng/mg creatinine (ng/mg Cre) and 8.1 ng/mg Cre, respectively.

Quantification of PGD₂ metabolites in urine and plasma has been reported. Early attempts at assay development centered on in vivo conversion of PGD₂ to 11β-PGF_{2α} by PGF synthase (Watanabe, K., Iguchi, Y., Iguchi, S. et al. Proc. Natl. Acad. Sci. USA, 83, 1986, 1583-1587). Measured concentrations of this metabolite in the urine of asthma patients by both gas chromatography-negative ion chemical ionization-mass spectrometry (GC-NICI-MS) and an enzyme immunoassay (EIA), however, was inconsistent (Bochenek, G., Nizankowska, E., Gielicz, A. et al. Thorax, 59, 2004, 459-464; Misso, N., Aggarwal, S., Phelps, S. et al. Clin. Exp. Allergy, 34, 2004, 624-631). Song, W. et al., reporting that tetranor-PGDM is an abundant biomarker of PGD₂ in human and murine urine, have quantified tetranor-PGDM levels using liquid chromatography-tandem mass spectrometry (HPLC-MS/MS) with ng/mg Cre detection limits.

Prostaglandin D antibodies in the art typically cross react with prostaglandin F-series and E-series counterparts. A need exists in the art for an immunoassay that specifically detects and measures tetranor-PGDM against PGF₂, metabolites, PGE₂ metabolites, and other PGD₂ metabolites in biological fluids for use in research, diagnostic, and clinical applications. The present invention meets this need. As will be evident elsewhere in this disclosure, an immunoassay that is capable of effectively detecting and measuring both tetranor-PGDM and the corresponding 9,10-dehydrate PGD₂ metabolite, tetranor-PGJM, to the exclusion of PGF₂ₐ metabolites, PGE₂ metabolites, and other PGD₂ metabolites may be used to accurately assess tetranor-PGDM levels and thus indirectly measure levels of PGD₂ biosynthesis. In addition to the likelihood that tetranor-PGJM is derived from dehydration of tetranor-PGDM (as prostaglandin J₂, or PGJ₂, is derived from dehydration of PGD₂), the electrophilic tetranor-PGJM is typically not detected in mammal urine (Ellis, C., Smigel, M., Oates, J. et al. J. Biol. Chem., 254, 1979, 4152-4163), likely due to rapid glutathione conjugation (Sanchez-Gomez, F., Gayarre, J., Avellano, M., Perez-Sala, D., Arch. Biochem. Biophys., 457, 2007, 150-159; Brunoldi, E., Zanoni, G., Vidari, G. et al. Chem. Res. Toxicol., 20, 2007, 1528-1535; Paumi, C., Smitherman, P.; Townsend, A., Morrow, C., Biochemistry, 43, 2004, 2345-2352).

### SUMMARY OF THE INVENTION

The present invention comprises tetranor-PGDM-carrier protein conjugates and tetranor-PGJM-carrier protein conjugates and methods for preparing them.

The present invention also comprises the use of tetranor-PGDM-carrier protein conjugates or tetranor-PGJM-carrier protein conjugates, acting as immunogens, for generating antibodies specific for tetranor-PGDM and/or tetranor-PGJM, as well as to the respective antibodies themselves.

The present invention also comprises tetranor-PGDM-molecular tag and tetranor-PGJM-molecular tag conjugates, each acting as a tracer that may be used in an assay for measuring concentration of tetranor-PGDM and/or tetranor-PGJM in a test sample.

The present invention also comprises assay kits used for measuring tetranor-PGDM and tetranor-PGJM metabolite levels in biological samples, wherein the assay kits comprise antibodies specific for tetranor-PGDM and tetranor-PGJM and a tracer comprising tetranor-PGDM and/or tetranor-PGJM covalently bonded to a molecular tag that produces a readable signal that may be measured to calculate concentration of tetranor-PGDM and/or tetranor-PGJM in a test sample.

The present invention also comprises a method for measuring tetranor-PGDM and tetranor-PGJM metabolite levels in biological samples utilizing assay kits comprising antibodies specific for tetranor-PGDM and tetranor-PGJM and a tracer comprising tetranor-PGDM and/or tetranor-PGJM covalently bonded to a molecular tag that produces a readable signal that may be measured to calculate concentration of tetranor-PGDM and/or tetranor-PGJM in a test sample.

Other exemplary embodiments of the invention will become apparent from the detailed description provided hereinafter. It should be understood that the detailed description and specific examples, while disclosing exemplary embodiments of the invention, are intended for purposes of illustration only and are not intended to limit the scope of the invention.

### BRIEF DESCRIPTION OF THE DRAWINGS

FIG. 1 is a linear plot showing correlation between concentrations of tetranor-PGDM added to urine samples and tetranor-PGDM measured by an exemplary embodiment described herein (Example 3). Urine samples were spiked with tetranor-PGDM, diluted as described in Example 3, Step B (Sample Preparation) and analyzed using the tetranor-PGDM EIA. The y-intercept corresponds to the amount of tetranor-PGDM in unspiked urine. Error bars represent standard deviations obtained from multiple dilutions of each sample.

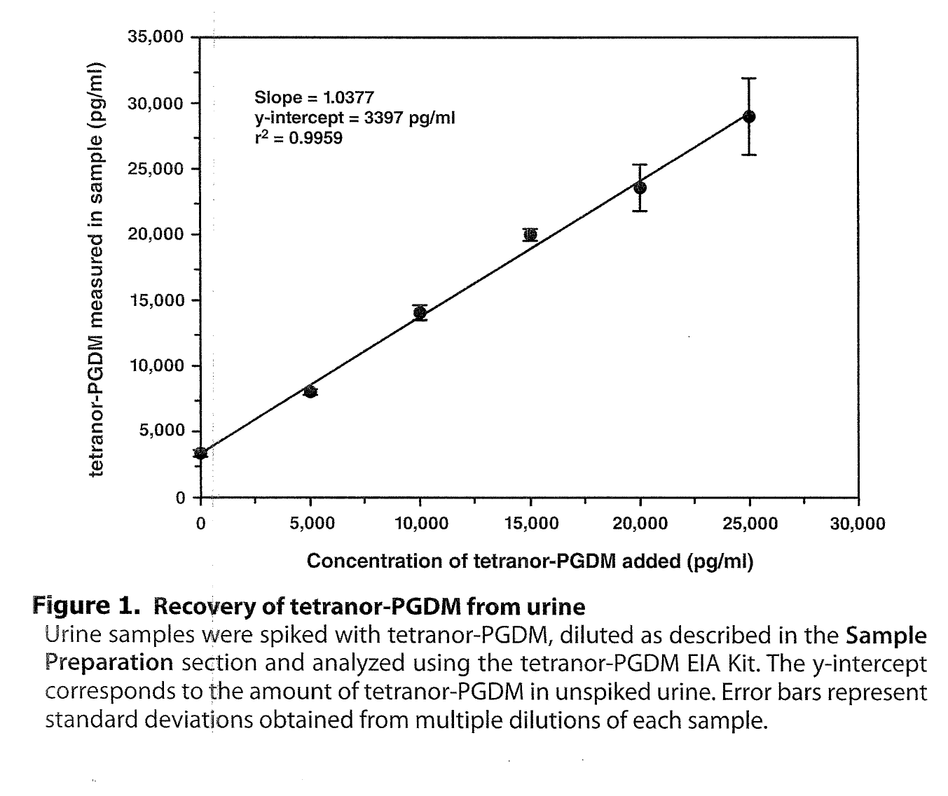

FIG. 2 illustrates the preparation of the tetranor-PGDM standards described below in Example 3, Step C1a.

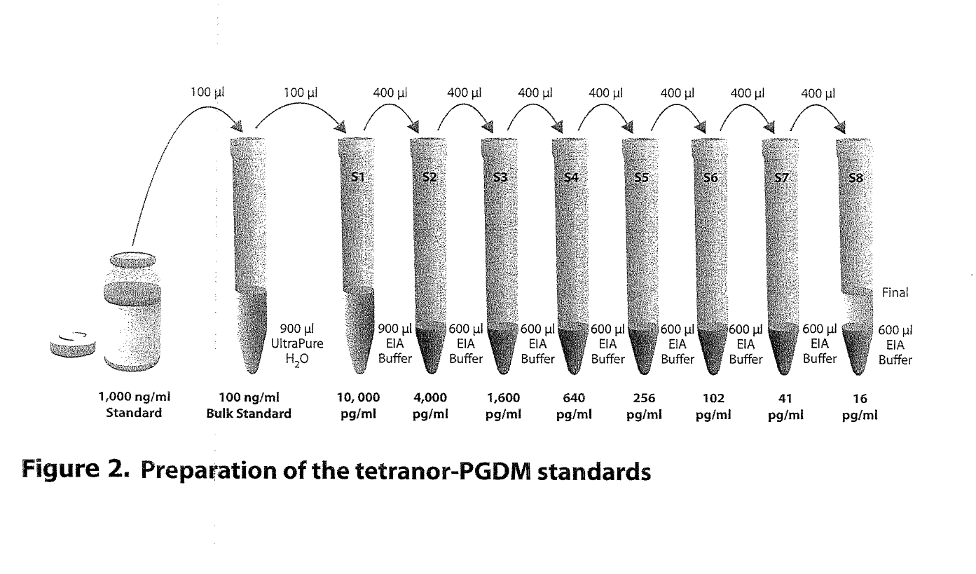

FIG. 3 Example 3, Step D2a shows a typical tetranor-PGDM EIA Standard curve.

### DETAILED DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances.

Unless otherwise defined herein, scientific and technical terms used in connection with the exemplary embodiments shall have the meanings that are commonly understood by those of ordinary skill in the art.

Further, unless otherwise required by context, singular terms shall include pluralities and plural terms shall include the singular. Generally, nomenclature used in connection with, and techniques of chemistry and biology described herein are those well known and commonly used in the art.

The term “alkyl,” alone or in combination, means an acyclic or cyclic radical, linear or branched, preferably containing from 1 to about 6 carbon atoms. Examples of such radicals include methyl, ethyl, n-propyl, isopropyl, n-butyl, isobutyl, sec-butyl, tert-butyl, pentyl, iso-amyl, hexyl, cyclopropyl, cyclopropylmethyl, cyclobutyl, cyclopentyl, cyclohexyl, and the like.

The term “tertiary amine base,” as used herein, refers to an amine comprising a nitrogen atom bearing a lone pair of electrons and three organic groups (substituting for the three hydrogen atoms of ammonia) that allow the nitrogen atom sufficient basicity to react with acidic hydrogen atoms of reactants or free solvated protons in a reaction mixture to form an ammonium salt comprising the nitrogen atom bearing a positive charge after forming a covalent bond with said acidic hydrogen or proton, the acidic hydrogen or proton, and the three organic groups. In certain embodiments, the organic groups comprise equivalent or various alkyl radicals as described above. In certain embodiments, the alkyl radicals are ethyl or isopropyl groups. In certain embodiments, the tertiary amine base is N,N-diisopropylethylamine, triethylamine, or triisopropylamine.

The term “biological fluids,” as used herein, refers to fluids that have human or animal origin, including but not limited to urine, whole blood, plasma, mucus, perspiration, saliva, semen, and vaginal fluid.

The term “Ellman's Reagent,” as used herein, refers to a product sold by Cayman Chemical Company, Incorporated (Catalog No. 400050) comprising 5,5′-dithiobis-(2-nitrobenzoic acid) (DTNB) and acetylthiocholine. The reagent is sold as a solid mixture and may be reconstituted into a solution by dissolving in a solvent such as water as described elsewhere in this disclosure.

The term “blank,” as used herein, refers to background absorbance caused by Ellman's Reagent. The blank absorbance should be subtracted from the absorbance readings of all other wells.

The term “total activity,” as used herein, refers to total enzymatic activity of an enzymatic tracer. This is analogous to the specific activity of a radioactive tracer.

The term “non-specific binding (NSB),” as used herein, refers to non-immunological binding of the tracer to the well. Even in the absence of specific antibody a very small amount of tracer still binds to the well; the NSB is a measure of this low binding.

The term “maximum binding (B₀),” as used herein, refers to the maximum amount of the tracer that the antibody can bind in the absence of free analyte.

The term “% bound/maximum bound (% B/B₀),” as used herein, refers to the ratio of the absorbance of a particular sample or standard well to that of the maximum binding (B₀) well.

The term “standard curve,” as used herein, refers to a plot of the % B/B₀ values versus concentration of a series of wells containing various known amounts of analyte.

The term “determination (dtn),” as used herein, refers to refers to an amount of reagent, where one dtn is the amount of reagent used per well.

The term “immunogen,” as used herein, refers to an antigen that induces adaptive immunity.

The term “antigen,” as used herein, refers to any molecule recognized by the immune system.

The term “adaptive immunity,” as used herein, refers to antigen-specific immune response.

The term “carrier protein,” as used herein, refers to a protein to which PGD₂ metabolite tetranor-PGDM or PGD₂ metabolite tetranor-PGJM is covalently attached to form a metabolite-carrier protein conjugate such that an immune response to tetranor-PGDM or tetranor-PGJM is generated when the conjugate is injected into a host organism. Exemplary carrier proteins include but are not limited to keyhole limpet hemocyanin (KLH), bovine serum albumin (BSA), ovalbumin, and thyroglobulin.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the exemplary embodiments may exist in a variety of forms including, for example, polyclonal antibodies, monoclonal antibodies, intracellular antibodies (“intrabodies”), Fv, Fab, and F(ab)₂, as well as single chain antibodies (scFv), heavy chain antibodies, such as camelid antibodies, and humanized antibodies (Harlow, E. and Lane, D., Editors, 1999, Using Antibodies: A Laboratory Manual, Cold Spring Harbor Laboratory Press, NY; Harlow, E. and Lane, D., Editors, 1988, Antibodies: A Laboratory Manual, Cold Spring Harbor Laboratory Press, NY; Huston, J., Levinson, D., Mudgett-Hunter, M. et al. Proc. Natl. Acad. Sci. USA, 85, 1988, 5879-5883; Bird, R., Hardman, K., Jacobson, J. et al. Science, 242, 1988, 423-426.).

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

The term “specifically binds,” as used herein with respect to an antibody, means an antibody which recognizes a specific antigen, but does not substantially recognize or bind other molecules in a sample. For example, in a sample containing tetranor-PGDM, an antibody that specifically binds to tetranor-PGDM recognizes and binds to tetranor-PGDM but does not substantially recognize or bind to other molecules in the sample. Furthermore, as used herein with respect to an antibody, the term “specifically binds” may also mean an antibody which recognizes a metabolite and a closely-related molecule derived from said metabolite. For example, an antibody may be considered to recognize and “specifically bind” to tetranor-PGDM and its closely-related dehydrate tetranor-PGJM while not substantially recognizing or binding to other molecules.

The term “detection analyte” as used herein, may be used interchangeably with the term “tracer” and refers to any entity comprising tetranor-PGDM or tetranor-PGJM and a molecular tag covalently linked to tetranor-PGDM or tetranor-PGJM, which produces a readable signal that may be measured to calculate concentration of the respective tetranor-PGDM or tetranor-PGJM in a test sample.

The term “molecular tag,” as used herein, is a molecule or molecular moiety such as, but not limited to, a fluorophore moiety, a chemiluminescent moiety, a biotin-avidin system, or a protein that catalyzes a conversion of a substrate of the protein into a product for which a measured readable signal or property of the test sample is changed by said conversion.

The exemplary embodiments described herein are useful in various applications, including but not limited to, research, diagnostic, and clinical.

The exemplary embodiments described herein may be based on the discovery of an antibody that specifically binds to tetranor-PGDM (I) and tetranor-PGJM (II).

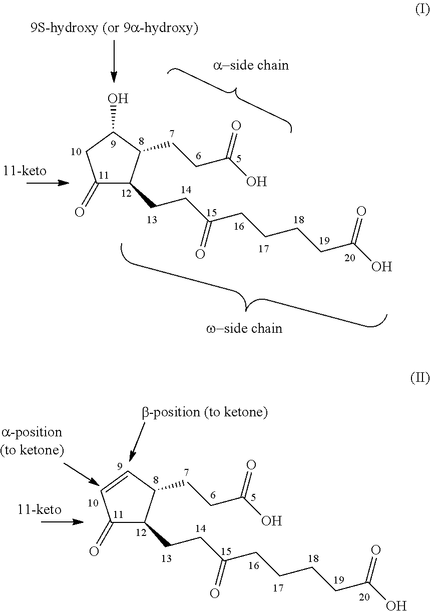

Tetranor-PGJM may be formed by dehydration of the β-hydroxyketone tetranor-PGDM, which involves the elimination of a water equivalent consisting of the 9S-hydroxy group at the keto β-position and an adjacent proton on the keto α-position, to afford the cyclopentenone tetranor-PGJM scaffold. Dehydration may occur spontaneously under certain conditions in an essentially irreversible manner.

Dehydration may also proceed in an acid- or base-catalyzed manner under certain conditions. Dehydration may also proceed through lactonization (internal cyclic ester formation) of tetranor-PGDM, which occurs by intramolecular reaction between the 9-hydroxy group and the carboxyl terminus of the α-side chain that, with concomitant loss of a water molecule, forms the internal cyclic ester, or δ-lactone (IV), followed by subsequent elimination/lactone ring-opening and proton transfer to provide tetranor-PGJM according to the following scheme:

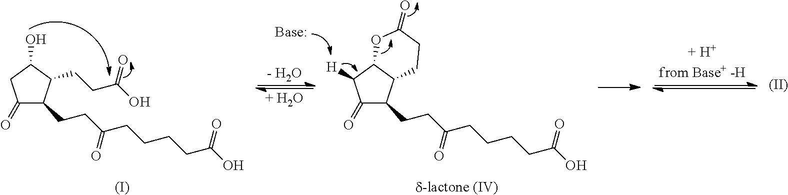

Tetranor-PGJM may also be formed by metabolism of PGD₂, a dehydrated metabolite of PGD₂, to tetranor-PGJM in a metabolic pathway similar to that of the metabolism of PGD₂ to tetranor-PGDM.

The α,β-unsaturated ketone (enone), tetranor-PGJM, is an electrophilic molecule (electrophile) that possesses the propensity to, by a Michael addition mechanism, chemically react with certain nucleophilic functional groups of nucleophilic molecules (nucleophiles), such as the sulfhydryl groups of glutathione (GSH) and protein cysteine (Cys) residues, to form tetranor-PGJM-derived PG metabolite moiety-nucleophile conjugates (IIIA) and (IIIB), also referred to herein as “tetranor-PGJM-nucleophile Michael adducts” as shown in the following schemes:

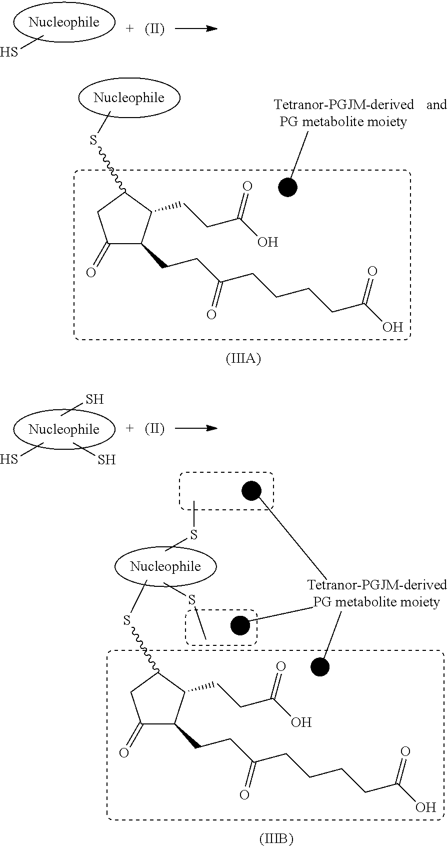

Exemplary embodiments may involve a sulfhydryl-bearing nucleophile comprising a carrier protein forming a Michael adduct with tetranor-PGJM to make an immunogen. Other exemplary embodiments may involve a sulfhydryl-bearing nucleophile comprising a molecular tag forming a Michael adduct with tetranor-PGJM for making a tracer. Exemplary tetranor-PGJM-nucleophile Michael adducts include but are not limited to tetranor-PGJM-carrier protein Michael adducts, tetranor-PGJM-enzyme Michael adducts, tetranor-PGJM-fluorophore Michael adducts, tetranor-PGJM-chemiluminescent moiety Michael adducts, and tetranor-PGJM-biotin-avidin system Michael adducts.

In certain embodiments, an immunogen or tracer wherein the prostaglandin metabolite is linked to the carrier protein or molecular tag, respectively, through one of the side chain terminal carboxyl moieties of the PG metabolite is produced by a conjugation reaction. Exemplary conjugation reactions may comprise forming a mixture comprising tetranor-PGDM wherein the mixture conditions cause dehydration of tetranor-PGDM starting material or tetranor-PGDM-nucleophile conjugate formed in the reaction mixture, resulting in the transformation of starting material tetranor-PGDM to tetranor-PGJM or the transformation of tetranor-PGDM-nucleophile conjugate to tetranor-PGJM-nucleophile conjugate. In these embodiments, the immunogen or tracer formed is enriched in tetranor-PGJM-immunogen/tracer conjugate. The practical effect of the dehydration in the formation of the immunogen or tracer may be negligible in that such conjugates may possess similar specificities against metabolites that are not either tetranor-PGDM or tetranor-PGJM and lack specificity for tetranor-PGDM over tetranor-PGJM or vice versa.

In particular, the present invention may first be directed to methods for preparing a tetranor-PGDM-carrier protein conjugate, a tetranor-PGJM-carrier protein conjugate, or a tetranor-PGJM-carrier protein Michael adduct. A tetranor-PGDM-carrier protein conjugate, as defined herein, is an immunogen that is capable of inducing the production of antibodies that specifically bind to tetranor-PGDM or that bind only to tetranor-PGDM and tetranor-PGJM when injected into a biological sample. Similarly, a tetranor-PGJM-carrier protein conjugate, as defined herein, is an immunogen that is capable of inducing the production of antibodies that specifically bind to tetranor-PGJM or that bind only to tetranor-PGDM and tetranor-PGJM when injected into a biological sample. Furthermore, a tetranor-PGJM-carrier protein Michael adduct, as defined herein, is an immunogen that is capable of inducing the production of antibodies that specifically bind to tetranor-PGDM, specifically bind to tetranor-PGJM, or that bind only to tetranor-PGDM and tetranor-PGJM when injected into a biological sample. The present invention also comprises the tetranor-PGDM-carrier protein conjugates, the tetranor-PGJM-carrier protein conjugates, and the tetranor-PGJM-carrier protein Michael adducts themselves.

In certain embodiments, the present invention may be directed to a method for preparing immunogens comprising tetranor-PGDM-KLH (“keyhole limpet hemocyanin”), tetranor-PGJM-KLH, tetranor-PGDM-BSA (“bovine serum albumin”), or tetranor-PGJM-BSA protein conjugates. The present invention also comprises tetranor-PGDM-KLH, tetranor-PGJM-KLH, tetranor-PGDM-BSA and tetranor-PGJM-BSA protein conjugates themselves.

In certain embodiments directed to methods for preparing a tetranor-PGDM-carrier protein conjugate or a tetranor-PGJM-carrier protein conjugate, the method comprises preparing a reaction mixture by contacting tetranor-PGDM or tetranor-PGJM with alkyl chloroformate and with a tertiary amine base. In certain of these embodiments, the alkyl group of the alky chloroformate comprises an acyclic or cyclic radical, linear or branched, preferably containing from 1 to about 6 carbon atoms. Examples of such radicals include methyl, ethyl, n-propyl, isopropyl, n-butyl, isobutyl, sec-butyl, tert-butyl, pentyl, iso-amyl, hexyl, cyclopropyl, cyclopropylmethyl, cyclobutyl, cyclopentyl, cyclohexyl, and the like. In certain of these embodiments, the alkyl chloroformate comprises isobutyl chloroformate. In certain of these embodiments, the tertiary amine base comprises N, N-diisopropylethylamine, triethylamine, or triisopropylamine.

In certain of these embodiments directed to methods for preparing a tetranor-PGDM-carrier protein conjugate or a tetranor-PGJM-carrier protein conjugate, the molar ratio of alkyl chloroformate with respect to tetranor-PGDM or tetranor-PGJM in the reaction mixture used to form the tetranor-PGDM-carrier protein conjugate or a tetranor-PGJM-carrier protein conjugate is from 10 to 200 mole percent, such as from 10 to 30 mole percent, such as 20 mole percent.

In certain of these embodiments directed to methods for preparing a tetranor-PGDM-carrier protein conjugate or a tetranor-PGJM-carrier protein conjugate, the molar ratio of tertiary amine base with respect to tetranor-PGDM or tetranor-PGJM in the reaction mixture used to form the tetranor-PGDM-carrier protein conjugate or a tetranor-PGJM-carrier protein conjugate is from 20 to 500 mole percent, such as from 300 to 400 mole percent, such as 380 mole percent.

In certain of these embodiments directed to methods for preparing a tetranor-PGDM-carrier protein conjugate or a tetranor-PGJM-carrier protein conjugate, the molar ratio of tetranor-PGDM or tetranor-PGJM to alkyl chloroformate (or isobutyl chloroformate) to tertiary amine base (i.e. tetranor-PGDM or tetranor-PGJM:alkyl chloroformate:tertiary amine base) in the reaction mixture used to form the tetranor-PGDM-carrier protein conjugate or a tetranor-PGJM-carrier protein conjugate is from 1:0.1:0.2 to 1:2:5; such as 1:1:1 or 1:0.2:3.8.

In one exemplary embodiment, a tetranor-PGDM-carrier protein conjugate or tetranor-PGJM-carrier protein conjugate may be formed according to a method comprising the following steps:
1. Preparing a reaction mixture by contacting an organic solution comprising 0.001-0.1 M tetranor-PGDM (limiting reagent) in a suitable organic solvent with an organic solution comprising alkyl chloroformate in said suitable organic solvent (10-200 mole % alkyl chloroformate with respect to the limiting reagent, 0.2-1 weight % alkyl chloroformate solute with respect to total mass of said organic solution comprising alkyl chloroformate) and with a tertiary amine base (20-500 mole % with respect to the limiting reagent) at a temperature range of −20° C. to +25° C.;
2. Mixing said reaction mixture at a temperature range of −5° C. to +25° C. for 1-4 hours;
3. Removing the solvent of said reaction mixture to provide a reaction mixture concentrate;
4. Adding a mixture comprising a carrier protein (0.001-0.2 mole % with respect to the limiting reagent, starting material tetranor-PGDM) and an aqueous buffer solution at pH range 7.2-7.6 to said reaction mixture concentrate to provide a second reaction mixture;
5. Stirring said second reaction mixture for 1-24 hours at +1 to +10° C. in the dark;
6. Purifying said second reaction mixture, such as by dialyzing said second reaction mixture through a 10,000 molecular weight (MW) cut-off membrane for 6-10 hours against an aqueous buffer solution comprising 0.05-0.2 M buffer, pH range 7.2-7.6 to provide a conjugate solution; and
7. Freezing aliquots of said conjugate solution at −15° C. to −40° C.

In another exemplary embodiment, a tetranor-PGDM-carrier protein conjugate or tetranor-PGJM-carrier protein conjugate may be formed according to a method comprising the following steps:
1. Preparing a reaction mixture by contacting a solution comprising 0.01-0.02 M tetranor-PGDM (limiting reagent) in acetonitrile with a solution comprising isobutyl chloroformate in acetonitrile (10-200 mole % isobutyl chloroformate with respect to the limiting reagent, 0.2-1 weight % isobutyl chloroformate solute with respect to total mass of said organic solution comprising isobutyl chloroformate) and with a tertiary amine base (20-500 mole % with respect to the limiting reagent) at a temperature range of −5° C. to +5° C.;
2. Mixing said reaction mixture at a temperature range of −5° C. to +5° C. for 1-4 hours;
3. Removing the solvent of said reaction mixture to provide a reaction mixture concentrate;
4. Adding a mixture comprising a carrier protein (0.001-0.1 mole % with respect to the limiting reagent, starting material tetranor-PGDM) and a 0.1 M aqueous potassium phosphate buffer solution at pH 7.4 to said reaction mixture concentrate to provide a second reaction mixture;
5. Stirring said second reaction mixture for 1-24 hours at +1 to +5° C. in the dark;
6. Purifying said second reaction mixture, such as by dialyzing said second reaction mixture through a 10,000 molecular weight (MW) cut-off membrane for 7-9 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGDM-carrier protein conjugate or tetranor-PGJM-carrier protein conjugate may be formed according to a method comprising the following steps:
1. Preparing a reaction mixture by contacting a solution comprising 0.01-0.02 M tetranor-PGDM in acetonitrile (limiting reagent) with a solution comprising isobutyl chloroformate in acetonitrile (10-200 mole % isobutyl chloroformate with respect to the limiting reagent, 0.2-1 weight % isobutyl chloroformate solute with respect to total mass of said organic solution comprising isobutyl chloroformate) and with a tertiary amine base (20-500 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGDM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGDM-carrier protein conjugate or tetranor-PGJM-carrier protein conjugate may be formed according to a method comprising the following steps:
1. Preparing a reaction mixture by contacting a solution comprising 0.01-0.02 M tetranor-PGDM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGDM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGDM-carrier protein conjugate or tetranor-PGJM-carrier protein conjugate may be formed according to a method comprising the following steps:
1. Preparing a reaction mixture by contacting a solution comprising 0.01-0.02 M tetranor-PGDM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGDM) comprising KLH, BSA, ovalbumin, or thyroglobulin, and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGDM-KLH conjugate or tetranor-PGJM-KLH conjugate may be formed according to a method comprising the following steps:
1. Preparing a reaction mixture by contacting a solution comprising 0.01-0.02 M tetranor-PGDM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising KLH (about 0.01 mole % with respect to the limiting reagent, starting material tetranor-PGDM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGDM-BSA conjugate or tetranor-PGJM-BSA conjugate may be formed according to a method comprising the following steps:
1. Preparing a reaction mixture by contacting a solution comprising 0.01-0.02 M tetranor-PGDM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising BSA (about 0.01 mole % with respect to the limiting reagent, starting material tetranor-PGDM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-carrier protein conjugate may be formed according to a method comprising the following steps:
1. Preparing a reaction mixture by contacting an organic solution comprising 0.001-0.1 M tetranor-PGJM (limiting reagent) in a suitable organic solvent with an organic solution comprising alkyl chloroformate in said suitable organic solvent (10-200 mole % alkyl chloroformate with respect to the limiting reagent, 0.2-1 weight % alkyl chloroformate solute with respect to total mass of said organic solution comprising alkyl chloroformate) and with a tertiary amine base (20-500 mole % with respect to the limiting reagent) at a temperature range of −20° C. to +25° C.;
2. Mixing said reaction mixture at a temperature range of −5° C. to +25° C. for 1-4 hours;
3. Removing the solvent of said reaction mixture to provide a reaction mixture concentrate;
4. Adding a mixture comprising a carrier protein (0.001-0.2 mole % with respect to the limiting reagent, starting material tetranor-PGJM) and an aqueous buffer solution at pH range 7.2-7.6 to said reaction mixture concentrate to provide a second reaction mixture;
5. Stirring said second reaction mixture for 1-24 hours at +1 to +10° C. in the dark;
6. Purifying said second reaction mixture, such as by dialyzing said second reaction mixture through a 10,000 molecular weight (MW) cut-off membrane for 6-10 hours against an aqueous buffer solution comprising 0.05-0.2 M buffer, pH range 7.2-7.6 to provide a conjugate solution; and
7. Freezing aliquots of said conjugate solution at −15° C. to −40° C.

In another exemplary embodiment, a tetranor-PGJM-carrier protein conjugate may be formed according to a method comprising the following steps:
1. Preparing a reaction mixture by contacting a solution comprising 0.01-0.02 M tetranor-PGJM (limiting reagent) in acetonitrile with a solution comprising isobutyl chloroformate in acetonitrile (10-200 mole % isobutyl chloroformate with respect to the limiting reagent, 0.2-1 weight % isobutyl chloroformate solute with respect to total mass of said organic solution comprising isobutyl chloroformate) and with a tertiary amine base (20-500 mole % with respect to the limiting reagent) at a temperature range of −5° C. to +5° C.;
2. Mixing said reaction mixture at a temperature range of −5° C. to +5° C. for 1-4 hours;
3. Removing the solvent of said reaction mixture to provide a reaction mixture concentrate;
4. Adding a mixture comprising a carrier protein (0.001-0.1 mole % with respect to the limiting reagent, starting material tetranor-PGJM) and a 0.1 M aqueous potassium phosphate buffer solution at pH 7.4 to said reaction mixture concentrate to provide a second reaction mixture;
5. Stirring said second reaction mixture for 1-24 hours at +1 to +5° C. in the dark;
6. Purifying said second reaction mixture, such as by dialyzing said second reaction mixture through a 10,000 molecular weight (MW) cut-off membrane for 7-9 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-carrier protein conjugate may be formed according to a method comprising the following steps:
1. Preparing a reaction mixture by contacting a solution comprising 0.01-0.02 M tetranor-PGJM in acetonitrile (limiting reagent) with a solution comprising isobutyl chloroformate in acetonitrile (10-200 mole % isobutyl chloroformate with respect to the limiting reagent, 0.2-1 weight % isobutyl chloroformate solute with respect to total mass of said organic solution comprising isobutyl chloroformate) and with a tertiary amine base (20-500 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGJM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-carrier protein conjugate may be formed according to a method comprising the following steps:
1. Preparing a reaction mixture by contacting a solution comprising 0.01-0.02 M tetranor-PGJM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGJM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-carrier protein conjugate may be formed according to a method comprising the following steps:
1. Preparing a reaction mixture by contacting a solution comprising 0.01-0.02 M tetranor-PGJM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising a carrier protein (about 0.01 mole % with respect to the limiting reagent, starting material tetranor-PGJM) comprising KLH, BSA, ovalbumin or thyroglobulin, and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-KLH conjugate may be formed according to a method comprising the following steps:
1. Preparing a reaction mixture by contacting a solution comprising 0.01-0.02 M tetranor-PGJM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising KLH (about 0.01 mole % with respect to the limiting reagent, starting material tetranor-PGJM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In another exemplary embodiment, a tetranor-PGJM-BSA conjugate may be formed according to a method comprising the following steps:
1. Preparing a reaction mixture by contacting a solution comprising 0.01-0.02 M tetranor-PGJM in acetonitrile (limiting reagent) with a solution comprising isobutyl chloroformate in acetonitrile (10-30 mole % isobutyl chloroformate with respect to the limiting reagent, 0.5-6 weight % isobutyl chloroformate solute with respect to total mass of said organic solution comprising isobutyl chloroformate) and with a tertiary amine base (300-400 mole % with respect to the limiting reagent) at 0° C.;
2. Mixing said reaction mixture at 0° C. for 2 hours;
3. Removing the solvent of said reaction mixture under a stream of nitrogen to provide a reaction mixture concentrate;
4. Adding a mixture comprising BSA (about 0.01 mole % with respect to the limiting reagent, starting material tetranor-PGJM) and a 0.1 M aqueous potassium phosphate buffer solution at pH 7.4 to said reaction mixture concentrate to provide a second reaction mixture;
5. Stirring said second reaction mixture for 12-18 hours at +4° C. in the dark;
6. Purifying said second reaction mixture, such as by dialyzing said second reaction mixture through a 10,000 molecular weight (MW) cut-off membrane for 8 hours against a 0.1 M aqueous potassium phosphate buffer solution at pH 7.4 to provide a conjugate solution; and
7. Freezing aliquots of said conjugate solution at −20° C.

In certain embodiments, the present invention may be directed to a method for preparing immunogens comprising tetranor-PGJM-KLH or tetranor-PGJM-BSA Michael adducts. The present invention also comprises tetranor-PGJM-KLH and tetranor-PGJM-BSA Michael adducts themselves.

In an exemplary embodiment, a tetranor-PGJM-carrier protein Michael adduct may be formed according to a method comprising the following steps:
1. Contacting a carrier protein in a suitable aqueous buffer with an excess of N-succinimidyl-S-acetylthioacetate in said aqueous buffer at a temperature range of +15° C. to +25° C.;
2. Mixing said reaction mixture at a temperature range of +15° C. to +25° C. for 0.25-2 hours to produce a derivatized carrier protein mixture;
3. Purifying said derivatized carrier protein mixture to prepare a purified derivatized carrier protein mixture;
4. Treating said purified derivatized carrier protein mixture with hydroxylamine to produce a sulfhydryl-deprotected carrier protein mixture;
5. Purifying said sulfhydryl-deprotected carrier protein mixture to prepare a purified sulfhydryl-deprotected carrier protein mixture;
6. Treating said purified sulfhydryl-deprotected carrier protein mixture with tetranor-PGJM to produce a tetranor-PGJM-carrier protein Michael adduct; and
7. Purifying said tetranor-PGJM-carrier protein Michael adduct to prepare a purified tetranor-PGJM-carrier protein Michael adduct.

In another exemplary embodiment, a tetranor-PGJM-KLH Michael adduct may be formed according to a method comprising the following steps:
1. Contacting KLH in a suitable aqueous buffer with an excess of N-succinimidyl-S-acetylthioacetate in said aqueous buffer at a temperature range of +15° C. to +25° C.;
2. Mixing said reaction mixture at a temperature range of +15° C. to +25° C. for 0.25-2 hours to produce a derivatized KLH mixture;
3. Purifying said derivatized KLH mixture to prepare a purified derivatized KLH mixture;
4. Treating said purified derivatized KLH mixture with hydroxylamine to produce a sulfhydryl-deprotected KLH mixture;
5. Purifying said sulfhydryl-deprotected KLH mixture to prepare a purified sulfhydryl-deprotected KLH mixture;
6. Treating said purified sulfhydryl-deprotected KLH mixture with tetranor-PGJM to produce a tetranor-PGJM-KLH Michael adduct; and
7. Purifying said tetranor-PGJM-KLH Michael adduct to prepare a purified tetranor-PGJM-KLH Michael adduct.

In another exemplary embodiment, a tetranor-PGJM-BSA Michael adduct may be formed according to a method comprising the following steps:
1. Contacting BSA in a suitable aqueous buffer with an excess of N-succinimidyl-S-acetylthioacetate in said aqueous buffer at a temperature range of +15° C. to +25° C.;
2. Mixing said reaction mixture at a temperature range of +15° C. to +25° C. for 0.25-2 hours to produce a derivatized BSA mixture;
3. Purifying said derivatized BSA mixture to prepare a purified derivatized BSA mixture;
4. Treating said purified derivatized BSA mixture with hydroxylamine to produce a sulfhydryl-deprotected BSA mixture;
5. Purifying said sulfhydryl-deprotected BSA mixture to prepare a purified sulfhydryl-deprotected BSA mixture;
6. Treating said purified sulfhydryl-deprotected BSA mixture with tetranor-PGJM to produce a tetranor-PGJM-BSA Michael adduct; and
7. Purifying said tetranor-PGJM-BSA Michael adduct to prepare a purified tetranor-PGJM-BSA Michael adduct.

The present invention may also be directed to the method of generating antibodies specific for tetranor-PGDM and tetranor-PGJM by immunizing a biological species (for example a mammal such as a mouse or rabbit), with a respective one of the tetranor-PGDM-canier protein conjugates, tetranor-PGJM-carrier protein conjugates, or tetranor-PGJM-carrier protein Michael adducts of the present invention described above. The protocols and methods for immunizing the biological species to generate the antibodies are done by methods well known to those of ordinary skill in the art in the fields of biochemistry and/or immunology. The tetranor-PGDM-carrier protein conjugate, tetranor-PGJM-carrier protein conjugate, or tetranor-PGJM-carrier protein Michael adduct is recognized by the biological species' adaptive immune system, thereby inducing the production of antibodies that specifically bind to tetranor-PGDM and tetranor-PGJM. The present invention also comprises the antibodies themselves, which may be monoclonal antibodies or polyclonal antibodies, depending upon the method utilized.

An exemplary embodiment may be directed to a method for preparing antibodies specific for tetranor-PGDM and tetranor-PGJM comprising an immunization step wherein a mammal is injected with an immunogen comprising a tetranor-PGDM-KLH conjugate or a tetranor-PGJM-KLH conjugate.

Another exemplary embodiment may be directed to a method for preparing antibodies specific for tetranor-PGDM and tetranor-PGJM comprising an immunization step wherein a mammal is injected with an immunogen comprising a tetranor-PGJM-KLH Michael adduct.

Another exemplary embodiment may be directed to a method for preparing antibodies specific for tetranor-PGDM and tetranor-PGJM comprising an immunization step wherein a mammal is injected with an immunogen comprising a tetranor-PGDM-BSA conjugate or a tetranor-PGJM-BSA conjugate.

Another exemplary embodiment may be directed to a method for preparing antibodies specific for tetranor-PGDM and tetranor-PGJM comprising an immunization step wherein a mammal is injected with an immunogen comprising a tetranor-PGJM-BSA Michael adduct.

Another exemplary embodiment may be directed to a method for preparing antibodies specific for tetranor-PGDM and tetranor-PGJM comprising an immunization step wherein a mouse is injected with an immunogen comprising a tetranor-PGDM-KLH conjugate or a tetranor-PGJM-KLH conjugate.

Another exemplary embodiment may be directed to a method for preparing antibodies specific for tetranor-PGDM and tetranor-PGJM comprising an immunization step wherein a mouse is injected with an immunogen comprising a tetranor-PGJM-KLH Michael adduct.

Another exemplary embodiment may be directed to a method for preparing antibodies specific for tetranor-PGDM and tetranor-PGJM comprising an immunization step wherein a mouse is injected with an immunogen comprising a tetranor-PGDM-BSA conjugate or a tetranor-PGJM-BSA conjugate.

Another exemplary embodiment may be directed to a method for preparing antibodies specific for tetranor-PGDM and tetranor-PGJM comprising an immunization step wherein a mouse is injected with an immunogen comprising a tetranor-PGJM-BSA Michael adduct.

Another exemplary embodiment may be directed to a method for preparing antibodies specific for tetranor-PGDM and tetranor-PGJM comprising an immunization step wherein a rabbit is injected with an immunogen comprising a tetranor-PGDM-KLH conjugate or a tetranor-PGJM-KLH conjugate.

Another exemplary embodiment may be directed to a method for preparing antibodies specific for tetranor-PGDM and tetranor-PGJM comprising an immunization step wherein a rabbit is injected with an immunogen comprising a tetranor-PGJM-KLH Michael adduct.

Another exemplary embodiment may be directed to a method for preparing antibodies specific for tetranor-PGDM and tetranor-PGJM comprising an immunization step wherein a rabbit is injected with an immunogen comprising a tetranor-PGDM-BSA conjugate or a tetranor-PGJM-BSA conjugate.

Another exemplary embodiment may be directed to a method for preparing antibodies specific for tetranor-PGDM and tetranor-PGJM comprising an immunization step wherein a rabbit is injected with an immunogen comprising a tetranor-PGJM-BSA Michael adduct.

In any of the above exemplary embodiments, the antibodies formed may be gathered from the biological sample for subsequent utilization in assay kits as described further below. The methods for gathering the antibodies are well known to those of ordinary skill in the art in the fields of biochemistry and/or immunology.

Still other exemplary embodiments may be directed methods for assessing biosynthesis of PGD₂ in a subject by measuring tetranor-PGDM and tetranor-PGJM levels derived from biological fluids taken from a subject. In certain embodiments, the assessment may be accomplished by measuring tetranor-PGDM metabolite levels and tetranor-PGJM metabolite levels in urine. In other embodiments, the assessment may be accomplished by measuring tetranor-PGDM metabolite levels and tetranor-PGJM metabolite levels in plasma.

In particular, the exemplary embodiments may be directed to competitive enzyme immunoassay (EIA) kits (assay kits) in which the competition between tetranor-PGDM or tetranor PGJM derived from the biological fluid of a subject and a constant concentration of detection analyte comprising tetranor-PGDM-molecular tag conjugate (tetranor-PGDM tracer), tetranor-PGJM-molecular tag conjugate (tetranor-PGJM tracer), or tetranor-PGJM-molecular tag Michael adduct tracer for a limited amount of tetranor-PGDM/tetranor-PGJM-specific antibody binding sites is measured. Kits may include a tracer comprising tetranor-PGDM or tetranor-PGJM covalently bound to a molecular tag such as acetylcholinesterase (AChE), horseradish peroxidase (HRP), alkaline phosphatase (AP), rhodamine, or fluorescein. Kits may further include a monoclonal or polyclonal antibody having reactivity specifically with tetranor-PGDM and tetranor-PGJM, including any of the antibodies formed in accordance with exemplary embodiments described above.

In an exemplary embodiment, a tracer may be formed according to a method comprising the following steps:
1. Chemically coupling tetranor-PGDM or tetranor-PGJM with an entity comprising a molecular tag that produces a readable signal that may be measured to calculate concentration of tetranor-PGDM and/or tetranor-PGJM in a test sample to produce a substance comprising a tracer; and
2. Purifying the substance comprising tracer to produce a substance comprising purified tracer.

In another exemplary embodiment, a tracer may be formed according to a method comprising the following steps:
1. Forming a chemically-activated tetranor-PGDM or tetranor-PGJM species;
2. Chemically coupling the activated species with an entity comprising a molecular tag that produces a readable signal that may be measured to calculate concentration of tetranor-PGDM and/or tetranor-PGJM in a test sample to produce a substance comprising a tracer; and
3. Purifying the substance comprising tracer to produce a substance comprising purified tracer.

In another exemplary embodiment, a tracer may be formed according to a method comprising the following steps:
1. Forming a mixture/solution comprising tetranor-PGDM, a tertiary amine base, and a suitable solvent;
2. Contacting the mixture/solution with a second solution/mixture comprising alkyl chloroformate and a suitable solvent to form a reaction mixture;
3. Removal of the solvent from the reaction mixture to obtain an activated tetranor-PGDM concentrate;
4. Reconstitution of the activated tetranor-PGDM concentrate in a solvent suitable for coupling the activated tetranor-PGDM with a molecular tag that produces a readable signal that may be measured to calculate concentration of tetranor-PGDM and/or tetranor-PGJM in a test sample to form an activated tetranor-PGDM solution;
5. Contacting the activated tetranor-PGDM solution with a mixture comprising an entity comprising a molecular tag that produces a readable signal that may be measured to calculate concentration of tetranor-PGDM and/or tetranor-PGJM in a test sample to produce a tracer mixture; and
6. Purifying the tracer mixture to produce a purified tracer mixture.

In another exemplary embodiment, a tracer may be formed according to a method comprising the following steps:
1. Forming a mixture comprising a suitable solvent, tetranor-PGDM, and 20-500 mole percent tertiary amine base with respect to the molar amount of the tetranor-PGDM;
2. Contacting the mixture with a second mixture comprising 10-200 mole percent alkyl chloroformate with respect to the molar amount of the tetranor-PGDM and a suitable solvent to form a reaction mixture;
3. Removal of the solvent from the reaction mixture to obtain an activated tetranor-PGDM concentrate;
4. Reconstitution of the activated tetranor-PGDM concentrate in a solvent suitable for coupling the activated tetranor-PGDM with a molecular tag that produces a readable signal that may be measured to calculate concentration of tetranor-PGDM and/or tetranor-PGJM in a test sample to form an activated tetranor-PGDM solution;
5. Contacting the activated tetranor-PGDM solution with a mixture comprising an entity comprising a molecular tag that produces a readable signal that may be measured to calculate concentration of tetranor-PGDM and/or tetranor-PGJM in a test sample to produce a tracer mixture; and
6. Purifying the tracer mixture to produce a purified tracer mixture.

In another exemplary embodiment, a tracer may be formed according to a method comprising the following steps:
1. Forming a mixture comprising a suitable solvent, tetranor-PGDM, and an equimolar amount of tertiary amine base with respect to the molar amount of the tetranor-PGDM;
2. Contacting the mixture with a second mixture comprising an equimolar amount of alkyl chloroformate with respect to the molar amount of the tetranor-PGDM and a suitable solvent to form a reaction mixture;
3. Removal of the solvent from the reaction mixture to obtain an activated tetranor-PGDM concentrate;
4. Reconstitution of the activated tetranor-PGDM concentrate in a solvent suitable for coupling the activated tetranor-PGDM with a molecular tag that produces a readable signal that may be measured to calculate concentration of tetranor-PGDM and/or tetranor-PGJM in a test sample to form an activated tetranor-PGDM solution;
5. Contacting the activated tetranor-PGDM solution with a mixture comprising an entity comprising a molecular tag that produces a readable signal that may be measured to calculate concentration of tetranor-PGDM and/or tetranor-PGJM in a test sample to produce a tracer mixture; and
6. Purifying the tracer mixture to produce a purified tracer mixture.

In another exemplary embodiment, a tracer may be formed according to a method comprising the following steps:
1. Forming a mixture comprising tetranor-PGDM, N,N-diisopropylethylamine, and acetonitrile;
2. Contacting the mixture with a second mixture comprising isobutyl chloroformate and acetonitrile to form a reaction mixture;
3. Removal of the acetonitrile from the reaction mixture to obtain an activated tetranor-PGDM concentrate;
4. Reconstitution of the activated tetranor-PGDM concentrate in N,N-dimethylformamide to form an activated tetranor-PGDM solution;
5. Contacting the activated tetranor-PGDM solution with a mixture comprising AChE and a suitable aqueous buffer solution to produce a tracer mixture; and
6. Purifying the tracer mixture on a size exclusion column to produce a purified tracer mixture.

In another exemplary embodiment, a tracer may be formed according to a method comprising the following steps:
1. Forming a mixture comprising acetonitrile and equimolar amounts of tetranor-PGDM and N,N-diisopropylethylamine;
2. Contacting the mixture with a second mixture comprising acetonitrile and an equimolar amount of isobutyl chloroformate with respect to the molar amount of the tetranor-PGDM to form a reaction mixture;
3. Removal of the acetonitrile from the reaction mixture to obtain an activated tetranor-PGDM concentrate;
4. Reconstitution of the activated tetranor-PGDM concentrate in N,N-dimethylformamide to form an activated tetranor-PGDM solution;
5. Contacting the activated tetranor-PGDM solution with a mixture comprising AChE and a suitable aqueous buffer solution to produce a tracer mixture; and
6. Purifying the tracer mixture on a size exclusion column to produce a purified tracer mixture.

In another exemplary embodiment, a tracer may be formed according to a method comprising the following steps:
1. Forming a mixture comprising tetranor-PGJM and a suitable solvent;
2. Forming a second mixture comprising an entity comprising a molecular tag that produces a readable signal that may be measured to calculate concentration of tetranor-PGDM and/or tetranor-PGJM in a test sample and a suitable solvent;
3. Combining the mixture with the second mixture to produce a reaction mixture; and
4. Purifying the reaction mixture to produce a purified tracer solution.

In another exemplary embodiment, a tracer may be formed according to a method comprising the following steps:
1. Forming a mixture comprising tetranor-PGJM and N,N-dimethyl formamide;
2. Forming a second mixture comprising AChE and a suitable aqueous buffer solution;
3. Combining the mixture with the second mixture to produce a reaction mixture; and
4. Purifying the reaction mixture on a size exclusion column to produce a purified tracer solution.

In another exemplary embodiment, a tracer may be formed according to a method comprising the following steps:
1. Contacting AChE in a suitable aqueous buffer with an excess of N-succinimidyl-S-acetylthioacetate (SATA) in said aqueous buffer at a temperature range of +15° C. to +25° C.;
2. Mixing said reaction mixture at a temperature range of +15° C. to +25° C. for 0.25-2 hours to produce a derivatized AChE mixture;
3. Purifying said derivatized carrier protein mixture to prepare a purified derivatized AChE mixture;
4. Treating said purified derivatized carrier protein mixture with hydroxylamine to produce a sulfhydryl-deprotected AChE mixture;
5. Purifying said sulfhydryl-deprotected AChE mixture to prepare a purified sulfhydryl-deprotected AChE mixture;
6. Treating said purified sulfhydryl-deprotected AChE mixture with tetranor-PGJM to produce a tetranor-PGJM-AChE Michael adduct; and
7. Purifying said tetranor-PGJM-AChE Michael adduct to prepare a purified tetranor-PGJM-AChE Michael adduct.

In another exemplary embodiment, a tracer may be formed according to a method comprising the following steps:
1. Contacting AChE in a suitable aqueous buffer with an excess of N-succinimidyl-S-acetylthiopropionate (SATP) in said aqueous buffer at a temperature range of +15° C. to +25° C.;
2. Mixing said reaction mixture at a temperature range of +15° C. to +25° C. for 0.25-2 hours to produce a derivatized AChE mixture;
3. Purifying said derivatized carrier protein mixture to prepare a purified derivatized AChE mixture;
4. Treating said purified derivatized carrier protein mixture with hydroxylamine to produce a sulfhydryl-deprotected AChE mixture;
5. Purifying said sulfhydryl-deprotected AChE mixture to prepare a purified sulfhydryl-deprotected AChE mixture;
6. Treating said purified sulfhydryl-deprotected AChE mixture with tetranor-PGJM to produce a tetranor-PGJM-AChE Michael adduct; and
7. Purifying said tetranor-PGJM-AChE Michael adduct to prepare a purified tetranor-PGJM-AChE Michael adduct.

The above description of exemplary embodiments, and examples provided below, are merely exemplary in nature and, thus, variations thereof are not to be regarded as a departure from the spirit and scope of the invention.

### EXAMPLES

The Examples provided herein describe embodiments directed to tetranor-PGDM-specific antibodies and immunogens used for generating antibodies specific for tetranor-PGDM and tetranor-PGJM. The Examples further provide methods for preparing antibodies that specifically bind tetranor-PGDM and tetranor-PGJM and immunogens used for generating antibodies specific for tetranor-PGDM and tetranor-PGJM. The Examples further describe tetranor-PGDM/tetranor-PGJM-molecular tag conjugate tracers, specifically tetranor-PGDM/tetranor-PGJM-enzyme (AChE) conjugate tracers, and provide methods for their preparation and use for quantifying tetranor-PGDM and tetranor-PGJM in test samples. The Examples further describe immunoassay kits and their use of tetranor-PGDM/tetranor-PGJM-specific antibodies for detecting and measuring quantities of tetranor-PGDM and tetranor-PGJM in a biological fluid.

### Example 1

### Monoclonal Methods

### Step A: Preparation of PGD Metabolite-Carrier Protein Immunogen

### Procedure 1: Preparation of Tetranor-PGDM/Tetranor-PGJM-Keyhole Limpet Hemocyanin (KLH) Immunogen (Immunogen 1)

Tetranor-PGDM (compound) was prepared by Cayman Chemical using a proprietary method. The identity of the compound was verified by mass spectrometry (MS) and by nuclear magnetic resonance (NMR) spectrometry. The compound was purified to >98% purity by preparatory thin layer chromatography (TLC), and purified compound (3 mg, 0.009 mmole) was dissolved in acetonitrile (600 μl) to provide a tetranor-PGDM solution (0.015 M). N,N-Diisopropylethylamine (density=0.742 g/mL, 6 μl) and isobutyl chloroformate in acetonitrile solution (0.54 weight %, density=1.044 g/mL, 50 μl) were added to the tetranor-PGDM solution.* This solution was mixed at 0° C. for two hours and was subsequently dried under a stream of nitrogen. Keyhole limpet hemocyanin (KLH) (5 mg, 0.01 mol % versus tetranor-PGDM with KLH MW 5,000,000) was dissolved in 0.1 M potassium phosphate (pH 7.4) (1 mL) and stirred overnight at 4° C. in the dark. The contents where then dialyzed through a 10,000 MW cut-off membrane for eight hours against 0.1 M potassium phosphate buffer, pH 7.4 (4 L) to provide an aqueous buffered conjugate solution. Aliquots of this solution were frozen at −20° C. for immunizations.

*Test reactions modeling the reaction conditions for forming the mixed anhydride of tetranor-PGDM followed by characterization of the product mixture by mass spectrometry showed that the major products were the dehydration product (tetranor-PGJM) and the tetranor-PGJM mono mixed anhydride conjugate. The highly specific anti-tetranor-PGDM/tetranor-PGJM versus tetranor-PGEM/tetranor-PGJM produced by immunization with the immunogen formed from tetranor-PGDM under these reaction conditions suggests the antibody recognizes the combination of a PGD or PGJ five-membered prostanoid ring with either the tetranor-α-chain or the 13,14-dihydro-15-keto-20-carboxy-ω-chain common to all tetranor-PG metabolites screened for cross-reactivity. None of the other PGs or PG metabolites screened for cross-reactivity with this antibody possesses either the α- or ω-chain of the tetranor metabolite species; therefore, their data shed no definitive light on whether conjugation of the tetranor-PG metabolites exists through the C1 or “C20” carboxylic acid.

### Procedure 1A: Preparation of Tetranor-PGDM/Tetranor-PGJM-Keyhole Limpet Hemocyanin (KLH) Immunogen (Immunogen 1A)

The method of Procedure 1 immediately above was used, except that equimolar amounts of tetranor-PGDM, isobutyl chloroformate, and N,N-diisopropylethylamine were combined, to form Immunogen 1A.

### Procedure 2: Preparation of Tetranor-PGJM-Bovine Serum Albumin (BSA) Immunogen (Immunogen 2) via Michael Addition

Bovine serum albumin (BSA, 3 mg) was mixed with a large molar excess of N-succinimidyl-S-acetylthioacetate (SATA, Pierce Protein Research Products/Thermo Scientific, Catalog No. 26102, 3 mg; alternatively, N-succinimidyl-S-acetylthiopropionate, or SATP, Thermo Scientific Catalog No. 26100, may be used with number of moles essentially equivalent to that of moles of SATA used) in aqueous potassium phosphate (KPhos) buffer pH 7.4 (100 mM, approximately 1 mL) and the mixture was incubated at room temperature for thirty minutes. The mixture was purified over a Sephadex G-25 size exclusion column equilibrated and run with an equilibrium mixture comprising KPhos buffer pH 7.4 (50 mM) and ethylenediaminetetraacetic acid (EDTA, 5 mM) to produce a purified acetylated BSA mixture. The purified acetylated BSA mixture was treated with hydroxylamine to a 50 mM concentration and the treated mixture was incubated at room temperature for two hours to produce a sulfhydryl-deprotected BSA mixture. The sulfhydryl-deprotected BSA mixture was purified over a Sephadex G-25 size exclusion column equilibrated and run with an equilibrium mixture comprising KPhos buffer pH 6 (50 mM) and EDTA (5 mM) to produce a set of fractions. Fractions were analyzed by absorbance at 280 nm or by the Bradford Method for protein quantification and fractions containing sufficient protein were combined to produce a purified sulfhydryl-deprotected BSA solution. A portion of the purified sulflaydryl-deprotected BSA solution containing 1 mg of the sulfhydryl-deprotected BSA solute was treated with tetranor-PGJM (1 mg) and the resulting mixture was incubated at 37° C. overnight and was purified over a Sephadex G-25 size exclusion column to produce a tetranor-PGJM-BSA Michael adduct conjugate (Immunogen 2). Conjugation efficiency was determined via immunoassay. A negative control was prepared by treating a portion of the purified sulfhydryl-deprotected BSA solution with N-ethylmaleimide (NEM) to block the sulfhydryl groups followed by incubation with tetranor-PGJM at 37° C. overnight. A much greater than one thousand-fold immunoreactivity was observed by Immunogen 2 over that of the negative control, supporting a thiol-specific conjugation.

### Step B: Preparation of Enzymatic Tracers

### Procedure 1: Preparation of Tetranor-PGDM-AChE Conjugate Via Mixed Anhydride Method (Tracer 1)

To a solution comprising tetranor-PGDM (50 μg) and acetonitrile (400 μL) chilled over ice was added a solution comprising N,N-diisopropylethylamine (19.7 μg) brought to 26.6 μL volume with acetonitrile. A solution comprising isobutylchloroformate (20.8 m) brought to 20 μL volume with acetonitrile was subsequently added and the reaction mixture was incubated on ice for two hours. The acetonitrile was blown off by a nitrogen stream while maintaining the mixture over ice and the concentrate was reconstituted with N,N-dimethylformamide (DMF, 200 μL). The reconstituted mixture was added to a mixture comprising acetylcholinesterase (AChE) (500 Units) and borate buffer pH 8.5 (100 mM, 1 mL) and the resulting combined mixture was incubated at 4° C. overnight. The mixture was purified on a G-25 Sephadex column eluting with 0.1 M potassium phosphate buffer, pH 7.4 and collecting 1-mL fractions. An aliquot (2 μL) of each fraction was added to a well of a 96-well plate, and each well was diluted with Ellman's Reagent (200 μL). Each diluted aliquot was incubated for about 30 seconds at room temperature and read at wavelength 414 nm. All fractions from which their corresponding aliquot-Ellman's mixtures produced greater than 10% of the maximum absorbance were combined. The combined fractions comprised concentrated bulk tracer solution, which was titered before use.

### Procedure 2: Preparation of Tetranor-PGDM-AChE Conjugate Via DCC Coupling Method (Tracer 2)

Anhydrous DMF was prepared by distillation and storage over molecular sieves. The dry DMF was used to prepare 10 mM solutions of N-hydroxysuccinimide (NHS), dicyclohexyldicarbodiimide (DCC), and tetranor-PGDM, each in a separate 10 mL reactivial that was oven dried and stored in a dessicator. In a new, dry 5 mL V-bottom reactivial, each of the prepared solutions (5 μl) was added, vortexed, briefly sealed with a septum cap and allowed to incubate at ambient temperature overnight. The next day, 0.1 M borate buffer (pH 8.5) (250 μl) was added, along with AChE (500 Units). The resulting mixture was incubated in the dark for 30 minutes at ambient temperature, then purified over a 30×1.5 cm Sephadex G-25 medium column and eluted with 0.1 M potassium phosphate buffer, pH 7.4. One-milliliter fractions were collected and those fractions with a positive Ellman's reaction were pooled. The tracer was diluted 1:1000 and tracer solution (50 μl) was used to detect specific antibody in 96-well microplate coated with mouse anti-rabbit immunoglobulin G (IgG).

### Procedure 3 (Michael Addition Method): Preparation of Tetranor-PGJM-AChE Michael Adduct (Tracer 3)

A mixture comprising tetranor-PGJM (50 m) and DMF (200 μL) was added to a mixture comprising AChE (500 Units) and KPhos buffer pH 7.4 (50 mM, 1 mL) and the combined mixtures were incubated at 37° C. overnight. The mixture was purified on a G-25 Sephadex column eluting with 0.1 M potassium phosphate buffer, pH 7.4 and collecting 1-mL fractions. An aliquot (2 μL) of each fraction was added to a well of a 96-well plate, and each well was diluted with Ellman's Reagent (200 μL). Each diluted aliquot was incubated for about 30 seconds at room temperature and read at wavelength 414 nm. All fractions from which their corresponding aliquot-Ellman's mixtures produced greater than 10% of the maximum absorbance were combined. The combined fractions comprised concentrated bulk tracer solution, which was titered before use.

*AChE contains 8 free thiols (sulfhydryls)/mole of tetramer if a higher level of conjugation is required more free thiols can be introduced via SATA or SATP modification according to embodiments described herein.

### Step C: Immunizations

Four to six week-old BALB/C mice (Charles River) were immunized by injecting intraperioneal (i.p.) with equal volumes of Immunogen 1A (100 μg; prepared in Step A, Procedure 1A of this Example) and Complete Freund's adjuvant followed by a boost 14 days later with an equivalent amount of immunogen in Incomplete Freund's adjuvant. Serum was collected on day 24 (10 days after the second immunization) and the titers determined based on immunoreactivity to tetranor-PGDM tracer (as described above). Mice with high serum titers to tetranor-PGDM were given a second boost i.p with antigen (100 μg) in Incomplete Freund's adjuvant on day 34 and serum was collected again and tested on day 44. Mice with high titers were given a final intravenous (i.v.) injection with immunogen (10 μg) in sterile saline. Three days later the mice were euthanized by carbon dioxide inhalation, their spleens removed under sterile conditions and prepared for fusion as described below.

### Step D: Splenocyte/Hybridoma Fusions

The fusion reagent was prepared by autoclaving PEG4000 (4.2 g) in a glass bottle and before the PEG4000 solidifies, add DMSO (1.5 mL) and bring the volume up to 10 mL with Dulbecco's PBS containing 0.1 mg/mL anhydrous CaCl₂ and 0.1 mg/mL MgCl₂ (hexahydrate). The reagent was stored at 4° C. Hypoxanthine Aminopterin Thymidine (HAT) selection medium was prepared by combining fetal bovine serum (50 mL), NCTS-109 (25 mL), Hypoxanthine/Thymidine (HT) solution (2.5 mL) (1.36 mg/mL hypoxanthine, 3.88 mg/mL thymidine), aminopterin (0.0018 mg/mL) (2.5 mL), 1-glutamine (2.5 mL), penicillin-streptomycin (2.5 mL), BM Condimed H1 (Roche) (25 mL) and bring the volume to 500 mL with RPMI-1640. HT growth medium was prepared similar to HAT medium with the omission of aminopterin, and the concentration of all other additives was doubled, except BM Condimed H1, which was reduced to 10 mL.

Immediately prior to fusion, approximately 3×10⁷ myeloma fusion partner cells (Ag8.653) were harvested and washed three times with RPMI-1640 by repeated centrifugation at 200×g and resuspension in RPMI-1640. The final pellet was resuspended in RPMI-1640 (10 mL) and the final cell number determined. A minimum of 20×10⁷ was required for fusion.

Spleens were aseptically excised from each mouse, rinsed with sterile RPMI 1640 medium (5 mL) in a P100 Petri dish, transferred to a second sterile dish, perfused with RPMI-1640 then minced with sterile forceps. The minced spleen suspension was transferred to a 15 mL conical tube to allow debris to settle. The cell suspension was transferred to a clean 15 mL tube, centrifuged at 200×g, the cell pellet resuspended in cold RPMI-1640 (10 mL) and the cell number determined.

For the fusion, the fusion ratio of splenocyte:myeloma cells was 5:1. (i.e. 10×10⁷ splenocytes to 2×10⁷ myeloma cells). The appropriate volumes of splenocytes and myeloma cells were transferred to a sterile 50 mL conical tube, centrifuged and the supernatant removed. The tube was heated in a 37° C. water bath for approximately one minute, and with continuous gentle swirling of the tube, fusion reagent (1.5 mL) was slowly added over the course of one minute. Over the course of an additional minute, warm RPMI-1640 (10 mL) was added, followed by centrifugation at 200×g to pellet the cells. The cell pellet was resuspended in HAT selection medium (125 mL) and 100 μl/well plated into each of twenty 96-well plates. The cells were incubated at 37° C. in a 5% CO₂ humidified atmosphere incubator. After 5-7 days, an additional HAT medium (100 μl) was added to each well. The plates were monitored until cell growth could be seen by direct observation. At this point the supernatants were harvested from wells with cell growth and screened for immunoreactivity with tetranor-PGDM tracer (see below).

### Step E: Hybridoma Screening System

Fusion supernatants were screened by transferring of supernatant (100 μl) into a well of a goat anti-mouse IgG coated plate from Cayman Chemical [Cat# 400009]. A negative control well contained HAT selection medium (100 μL) and a positive control well contained diluted immune mouse serum. Plates were incubated for 18 hours at room temperature and washed five times with PBS. Next 100 μL of tetranor-PGDM-AChE Tracer (Cayman Cat# 401000) was added to all wells. The plates were incubated for an additional two hours at room temperature, washed, and Ellman's reagent (200 μL) added to each well. The ODs were read at 415 nm at 30, 60, and 90 minute intervals. Wells with an elevated absorbance were considered positive for antibody production, and were expanded for further characterization.

Cells from wells that test positive in the first round of screening were transferred to HAT selection medium (3 mL) in wells of a 6-well dish or into HAT medium (5 mL) in a T25 flask. After vigorous cell growth was observed, a small volume of supernatant was harvested and re-screened as described above to eliminate any original false positives. Parental cultures that tested positive two or more times were expanded in T25 flasks and a portion cyropreserved (1-2 vials per parental) in liquid nitrogen. The remaining cells were maintained in culture for further screening and subcloning. In the next screen the parental were titered based on signal. The next screen consisted of a sensitivity screen selecting for antibody (Ab) with a suitable detection limit. The next screen excluded Ab with unacceptably high cross-reactivity (experiment described below) with parent metabolites, tetranor-PGFM, and tetranor-PGEM. The final screen consists of urinary measurements to confirm normal biological levels of tetranor-PGDM, and recovery experiments.

### Example 2

### Polyclonal Methods

### Step A: Preparation of PGD Metabolite-Carrier Protein Immunogen

### Procedure 1: Preparation of Tetranor-PGDM/Tetranor-PGJM-Keyhole Limpet Hemocyanin (KLH) Immunogen (Immunogen 1)

Tetranor-PGDM (compound) was prepared by Cayman Chemical using a proprietary method. The identity of the compound was verified by mass spectrometry (MS) and by nuclear magnetic resonance (NMR) spectrometry. The compound was purified to >98% purity by preparatory thin layer chromatography (TLC), and purified compound (3 mg, 0.009 mmole) was dissolved in acetonitrile (600 μl) to provide a tetranor-PGDM solution (0.015 M). N,N-Diisopropylethylamine (density=0.742 g/mL, 6 μl) and isobutyl chloroformate in acetonitrile solution (0.54 weight %, density=1.044 g/mL, 50 were added to the tetranor-PGDM solution.

*This solution was mixed at 0° C. for two hours and was subsequently dried under a stream of nitrogen. Keyhole limpet hemocyanin (KLH) (5 mg, 0.01 mol % versus tetranor-PGDM with KLH MW 5,000,000) was dissolved in 0.1 M potassium phosphate (pH 7.4) (1 mL) and stirred overnight at 4° C. in the dark. The contents where then dialyzed through a 10,000 MW cut-off membrane for eight hours against 0.1 M potassium phosphate buffer, pH 7.4 (4 L) to provide an aqueous buffered conjugate solution. Aliquots of this solution were frozen at −20° C. for immunizations.

Procedure 2: Preparation of Tetranor-PGJM-Bovine Serum Albumin (BSA) Immunogen (immunogen 2) Via Michael Addition

Bovine serum albumin (BSA, 3 mg) was mixed with a large molar excess of N-succinimidyl-S-acetylthioacetate (SATA, Pierce Protein Research Products/Thermo Scientific, Catalog No. 26102, 3 mg; alternatively, N-succinimidyl-S-acetylthiopropionate, or SATP, Thermo Scientific Catalog No. 26100, may be used with number of moles essentially equivalent to that of moles of SATA used) in aqueous potassium phosphate (KPhos) buffer pH 7.4 (100 mM, approximately 1 mL) and the mixture was incubated at room temperature for thirty minutes. The mixture was purified over a Sephadex G-25 size exclusion column equilibrated and run with an equilibrium mixture comprising KPhos buffer pH 7.4 (50 mM) and ethylenediaminetetraacetic acid (EDTA, 5 mM) to produce a purified acetylated BSA mixture. The purified acetylated BSA mixture was treated with hydroxylamine to a 50 mM concentration and the treated mixture was incubated at room temperature for two hours to produce a sulfhydryl-deprotected BSA mixture. The sulfhydryl-deprotected BSA mixture was purified over a Sephadex G-25 size exclusion column equilibrated and run with an equilibrium mixture comprising KPhos buffer pH 6 (50 mM) and EDTA (5 mM) to produce a set of fractions. Fractions were analyzed by absorbance at 280 nm or by the Bradford Method for protein quantification and fractions containing sufficient protein were combined to produce a purified sulfhydryl-deprotected BSA solution. A portion of the purified sulfhydryl-deprotected BSA solution containing 1 mg of the sulfhydryl-deprotected BSA solute was treated with tetranor-PGJM (1 mg) and the resulting mixture was incubated at 37° C. overnight and was purified over a Sephadex G-25 size exclusion column to produce a tetranor-PGJM-BSA Michael adduct conjugate (Immunogen 2). Conjugation efficiency was determined via immunoassay. A negative control was prepared by treating a portion of the purified sulfhydryl-deprotected BSA solution with N-ethylmaleimide (NEM) to block the sulfhydryl groups followed by incubation with tetranor-PGJM at 37° C. overnight. A much greater than one thousand-fold immunoreactivity was observed by Immunogen 2 over that of the negative control, supporting a thiol-specific conjugation.

### Step B: Preparation of Enzymatic Tracers

### Procedure 1: Preparation of Tetranor-PGDM-AChE Conjugate Via Mixed Anhydride Method (Tracer 1)

To a solution comprising tetranor-PGDM (50 μg) and acetonitrile (400 μL) chilled over ice was added a solution comprising N,N-diisopropylethylamine (19.7 μg) brought to 26.6 μL volume with acetonitrile. A solution comprising isobutylchloroformate (20.8 μg) brought to 20 μL volume with acetonitrile was subsequently added and the reaction mixture was incubated on ice for two hours. The acetonitrile was blown off by a nitrogen stream while maintaining the mixture over ice and the concentrate was reconstituted with N,N-dimethylformamide (DMF, 200 μL). The reconstituted mixture was added to a mixture comprising acetylcholinesterase (AChE) (500 Units) and borate buffer pH 8.5 (100 mM, 1 mL) and the resulting combined mixture was incubated at 4° C. overnight. The mixture was purified on a G-25 Sephadex column eluting with 0.1 M potassium phosphate buffer, pH 7.4 and collecting 1-mL fractions. An aliquot (2 μL) of each fraction was added to a well of a 96-well plate, and each well was diluted with Ellman's Reagent (200 μL). Each diluted aliquot was incubated for about 30 seconds at room temperature and read at wavelength 414 nm. All fractions from which their corresponding aliquot-Ellman's mixtures produced greater than 10% of the maximum absorbance were combined. The combined fractions comprised concentrated bulk tracer solution, which was titered before use.

### Procedure 2: Preparation of Tetranor-PGDM-AChE Conjugate Via DCC Coupling Method (Tracer 2)

Anhydrous DMF was prepared by distillation and storage over molecular sieves. The dry DMF was used to prepare 10 mM solutions of N-hydroxysuccinimide (NHS), dicyclohexyldicarbodiimide (DCC), and tetranor-PGDM, each in a separate 10 mL reactivial that was oven dried and stored in a dessicator. In a new, dry 5 mL V-bottom reactivial, each of the prepared solutions (5 μl) was added, vortexed, briefly sealed with a septum cap and allowed to incubate at ambient temperature overnight. The next day, 0.1 M borate buffer (pH 8.5) (250 μl) was added, along with AChE (500 Units). The resulting mixture was incubated in the dark for 30 minutes at ambient temperature, then purified over a 30×1.5 cm Sephadex G-25 medium column and eluted with 0.1 M potassium phosphate buffer, pH 7.4. One-milliliter fractions were collected and those fractions with a positive Ellman's reaction were pooled. The tracer was diluted 1:1000 and tracer solution (50 μl) was used to detect specific antibody in 96-well microplate coated with mouse anti-rabbit immunoglobulin G (IgG).

### Procedure 3 (Michael Addition Method): Preparation of Tetranor-PGJM-AChE Michael Adduct (Tracer 3)

A mixture comprising tetranor-PGJM (50 μg) and DMF (200 μL) was added to a mixture comprising AChE (500 Units) and KPhos buffer pH 7.4 (50 mM, 1 mL) and the combined mixtures were incubated at 37° C. overnight. The mixture was purified on a G-25 Sephadex column eluting with 0.1 M potassium phosphate buffer, pH 7.4 and collecting 1-mL fractions. An aliquot (2 of each fraction was added to a well of a 96-well plate, and each well was diluted with Ellman's Reagent (200 μL). Each diluted aliquot was incubated for about 30 seconds at room temperature and read at wavelength 414 nm. All fractions from which their corresponding aliquot-Ellman's mixtures produced greater than 10% of the maximum absorbance were combined. The combined fractions comprised concentrated bulk tracer solution, which was titered before use.

*AChE contains 8 free thiols (sulfhydryls)/mole of tetramer if a higher level of conjugation is required more free thiols can be introduced via SATA or SATP modification according to embodiments described herein.

### Step C: Immunizations

Rabbit immunizations were performed by Robert Sargeant Antibodies (655 Ash Street, Ramona, Calif. 92065) as follows: Male New Zealand White Rabbits 9-10 weeks of age, were immunized with Complete Freund's Adjuvant (CFA) initially, followed by Incomplete Freund's Adjuvant (IFA) for all subsequent injections. Immunogen 1 (200 μg; prepared in Step A, Procedure 1 of this Example) was injected for the first immunization and 100 μg for boosts. The immunogen was diluted to one milliliter with sterile saline and combined with one milliliter of the appropriate adjuvant. The antigen and adjuvant were mixed thoroughly to form a stable emulsion which is injected subcutaneously. Blood was collected from the central ear artery and allowed to clot and retract. The serum was decanted and clarified by centrifugation before freezing and subsequent shipment to Cayman Chemical.

### Step D: Initial Antisera Screening

Antisera (AS) from multiple bleeds from multiple rabbits were screened by titration on a 96-well microplate coated with mouse anti-rabbit IgG with detection of specific Ab based on its ability to bind the tetranor-PGDM-AChE conjugate (diluted 1:1000). The next screen consisted of a sensitivity screen selecting for AS with a suitable detection limit. The next screen excluded AS with unacceptably high cross-reactivity (experiment described below) with parent metabolites, tetranor-PGFM, and tetranor-PGEM. The final screen consisted of urinary measurements to confirm normal biological levels of tetranor-PGDM, and recovery experiments. Only one AS passed all screens.

### Step E: Cross-Reactivity Testing

To determine cross-reactivity of an antibody, the standard curve using optimal tracer and Ab dilutions according the kit instructions was set up. Tetranor PGDM was diluted to 10 ng/mL for the first point in the standard curve and diluted 2.5-fold serially 7 times. First standard dilutions (10 μg/mL) of tetranor-PGJM, tetranor-PGEM, tetranor-PGFM, PGA₂, PGD₂, PGE₂, PGF_{2α}, PGJ₂, 13,14-dihydro-15-keto PGD₂, 13,14-dihydro-15-keto-PGE₂, 13,14-dihydro-15-keto-PGF₂, were prepared and 7 additional 6-fold serial dilutions were performed. IC₅₀s of all compounds were determined and each percent cross-reactivity (% XR) values of the potentially cross-reactive compounds was determined by dividing the IC₅₀ of tetranor-PGDM by the IC₅₀ of the test compound and multiplying by 100. See the chart below for determined % XR values.

| Compound | % XR |
|---|---|
| tetranor-PGJM | 376% |
| tetranor-PGDM | 100% |
| tetranor-PGEM | 0.3% |
| tetranor-PGFM | <0.01% |
| Prostaglandin A₂ | <0.01% |
| Prostaglandin D₂ | <0.01% |
| 13,14-dihydro-15-keto Prostaglandin D₂ | <0.01% |
| Prostaglandin E2 | <0.01% |
| 13,14-dihydro-15-keto Prostaglandin E₂ | <0.01% |
| Prostaglandin F_{2α} | <0.01% |
| 13,14-dihydro-15-keto Prostaglandin F_{2α} | <0.01% |
| Prostaglandin J₂ | <0.01% |

### Example 3

### Quantification of Tetranor-PGDM in Urine

### Step A: Buffer Preparation

### 1. EIA Buffer Preparation

The contents of one vial of EIA Buffer Concentrate (10×) (Cayman Chemical Company, Incorporated Catalog No. 400060) was diluted with UltraPure water (Cayman Chemical Company, Incorporated Catalog No. 400000). The vial was rinsed to remove any salts that may have precipitated.

### 2. Wash Buffer Preparation

The Wash Buffer Concentrate (400×) (5 mL, 96-well kit; Cayman Chemical Company, Incorporated Catalog No. 400062) was diluted with UltraPure water to a total volume of 2 liters and Tween 20 (1 mL, Cayman Chemical Company, Incorporated Catalog No. 400035). Alternatively, the Wash Buffer Concentrate (400×) (12.5 mL, 480-well kit; Cayman Chemical Company, Incorporated Catalog No. 400062) was diluted with UltraPure water to a total volume of 5 liters and Tween 20 (2.5 mL, Cayman Chemical Company, Incorporated Catalog No. 400035). Smaller volumes of Wash Buffer can be prepared by diluting the Wash Buffer Concentrate 1:400 and adding Tween 20 (0.5 mL/liter of Wash Buffer).

### Step B: Sample Preparation

This assay has been validated for urine samples (diluted at least 1:2).

Proper sample storage and preparation are essential for consistent and accurate results. PGD₂ is chemically unstable in biological samples, especially those containing albumin (Fitzpatrick, F. and Wynalda, M. J. Biol. Chem., 258, 1983, 11713-11718).

All samples must be free of organic solvents prior to assay.

Samples should be assayed immediately after collection; samples that cannot be assayed immediately should be stored at −80° C.

Samples of rabbit origin may contain antibodies which interfere with the assay by binding to the mouse anti-rabbit IgG-coated plate. All rabbit samples should be purified prior to use in the assay.

Urinary concentrations of tetranor-PGDM vary considerably and, as with any urinary marker, the values obtained by EIA should be standardized to creatinine levels.

### Step C: Assay Protocol

### Step C1: Preparation of Assay-Specific Reagents

### Step C1a: Tetranor-PGDM EIA Standard

The tetranor-PGDM EIA Standard (100 μl, Cayman Chemical Company, Incorporated Catalog No. 401004) was transferred into a clean test tube and diluted with UltraPure water (900 μl). The concentration of this solution (the bulk standard) was 100 ng/mL. (If assaying culture medium samples that have not been diluted with EIA Buffer, culture medium should be used in place of EIA Buffer for dilution of the standard curve.

Eight clean test tubes were numbered #1 through #8. EIA Buffer (900 μl) was aliquoted to tube #1 and 600 μl of EIA Buffer to tubes #2-8. Bulk standard (100 μl) was transferred to tube #1 and the contents of the tube were mixed thoroughly. The standard was serially diluted by removing 400 μl from tube #1 and placing in tube #2; the contents of tube #2 were subsequently mixed thoroughly. Contents from tube #2 (400 μl) was transferred to tube #3; the contents of tube #3 were subsequently mixed thoroughly. This process was repeated for tubes #4-8. (These diluted standards should not be stored for more than 24 hours).

### Step C1b: Tetranor-PGDM AChE Tracer

The tetranor-PGDM AChE Tracer was reconstituted as follows. First, Tetranor-PGDM AChE Tracer (100 dtn, 96-well kit; Cayman Chemical Company, Incorporated Catalog No. 401000) was reconstituted with EIA Buffer (6 mL). Alternatively, Tetranor-PGDM AChE Tracer (500 dtn, 480-well kit; Cayman Chemical Company, Incorporated Catalog No. 401000) was reconstituted with EIA Buffer (30 mL).

The reconstituted tetranor-PGDM AChE Tracer should be stored at 4° C. (do not freeze) and used within four weeks. A tracer dye may be added to the tracer to aid in visualization of tracer-containing wells (not required). The dye is added to the reconstituted tracer at a final dilution of 1:100 (60 μl of dye is added to 6 mL of tracer, or 300 μl of dye is added to 30 mL of tracer).

### Step C1c: Tetranor-PGDM EIA Antiserum

The tetranor-PGDM EIA Antiserum was reconstituted as follows: First, Tetranor-PGDM EIA Antiserum (100 dtn, 96-well kit; Cayman Chemical Company, Incorporated Catalog No. 401002) was reconstituted with EIA Buffer (6 mL). Alternatively, Tetranor-PGDM EIA Antiserum (500 dtn, 480-well kit; Cayman Chemical Company, Incorporated Catalog No. 401002) was reconstituted with EIA Buffer (30 mL).

The reconstituted tetranor-PGDM EIA Antiserum should be stored at 4° C. and used within four weeks. An antiserum dye may be added to the antiserum to aid in visualization of antiserum-containing wells (not required). The dye is added to the reconstituted antiserum at a final dilution of 1:100 (60 μl of dye is added to 6 mL of antiserum or 300 μl of dye is added to 30 mL of antiserum).

### Step C2: Plate Setup

Each 96-well plate or set of strips contain a minimum of two blanks (Blk), two non-specific binding wells (NSB), two maximum binding wells (B₀), and an eight point standard curve run in duplicate. Each assay is assayed at two dilutions and each dilution is assayed in duplicate or triplicate.

### Step C3: Performing the Assay

### Step C3a: Addition of the Reagents:

1. EIA Buffer: EIA Buffer is added to NSB wells (100 col) and B₀ wells_(50 μl). If culture medium is used to dilute the standard curve, culture medium (50 μl) is substituted for EIA Buffer in the NSB and B₀ wells (i.e. 50 μl of culture medium is added to NSB and B₀ wells and 50 μl of EIA Buffer to NSB wells.).

2. Tetranor-PGDM EIA Standard: 50 μl from tube #8 is added to both of the lowest standard wells (S8). 50 μl from tube #7 is added to each of the next two standard wells (S7). This procedure is continued until all of the standards are aliquoted.

3. Samples: 50 μl of sample is added per well. Each sample is assayed at a minimum of two dilutions. Each dilution is assayed in duplicate or triplicate.

4. Tetranor-PGDM ACNE Tracer: 50 μl is added to each well except the total activity (TA) and the Blank (Bik) wells.

5. Tetranor-PGDM EIA Antiserum: 50 μl is added to each well except the TA, the NSB, and the Bik wells.

Step C3b: Incubation of the Plate: Each Plate is Covered with Plastic Film and Incubated Overnight at 4° C.

### Step C3c: Development of the Plate

1. Ellman's Reagent (100 dtn vial for 96-well kit) was reconstituted with UltraPure water (20 mL). Alternatively, Ellman's Reagent (250 dtn vial for 480-well kit) was reconstituted with UltraPure water (50 mL).

2. The wells were emptied and rinsed five times with Wash Buffer.

3. Ellman's Reagent (200 μl) was added to each well.

4. Tracer (5 μl) was added to the Total Activity wells.

5. The plate was covered with plastic film. Optimum development was obtained by using an orbital shaker equipped with a large, flat cover to allow the plates to develop in the dark for 60-90 minutes.

### Step C3d: Reading the Plate

1. The bottom of the plate was wiped with a clean tissue.

2. The plate cover was removed.
3. The plate was read at a wavelength between 405 and 420 nm. The absorbance was checked periodically until the B₀ wells had reached a minimum of 0.3 A.U. (blank subtracted). The plate was read when the absorbance of the B₀ wells were in the range of 0.3-1.0 A.U. (blank subtracted).

### Step D: Analysis

The data was plotted as % B/B₀ versus log concentration using either a 4-parameter logistic or log-logit curve fit.

### Step D1: Calculations

Step D1a: Preparation of the Data (Absorbance Reading of the Blank Wells Were Subtracted from the Absorbance Readings of the Rest of the Plate if not Done Automatically be the Plate Reader).

1. Absorbance readings from the NSB wells were averaged.

2. Absorbance readings from the B₀ wells were averaged.

3. The NSB average was subtracted from the B₀ average. This is the corrected B₀ or corrected maximum binding.

4. The % B/B₀ was calculated for the remaining wells. (The average NSB absorbance was subtracted from the S1 absorbance and divided by the corrected B₀ from step 3 immediately above. This value was multiplied by 100 to obtain % B/B₀ and the calculation was repeated for S2-S8 and all sample wells.)

### Step D1b: Plot of the Standard Curve

The % B/B₀ was plotted for standards S1-S8 versus tetranor-PGDM concentration using linear (y) and log(x) axes and the data was fit to a 4-parameter logistic equation.

Alternative plot: the data may also be linearized using a logit transformation. The equation for this conversion is:

logit(B/B₀)=ln [B/B₀/(1−B/B₀)].

The data is plotted as logit (B/B₀) versus log concentrations and a linear regression fit is performed.

### Step D1c: Determination of the Sample Concentration

The % B/B₀ is calculated for each sample. The concentration of each sample is determined using the equation obtained from the standard curve plot. Samples with % B/B₀ values greater than 80% or less than 20% should be re-assayed as they generally fall outside the range of the standard curve. A 20% or greater disparity between the apparent concentrations of two different dilutions of the same sample indicate interference which is eliminated by purification.

### Step D2: Performance Characteristics

### Step D2a: Sample Data

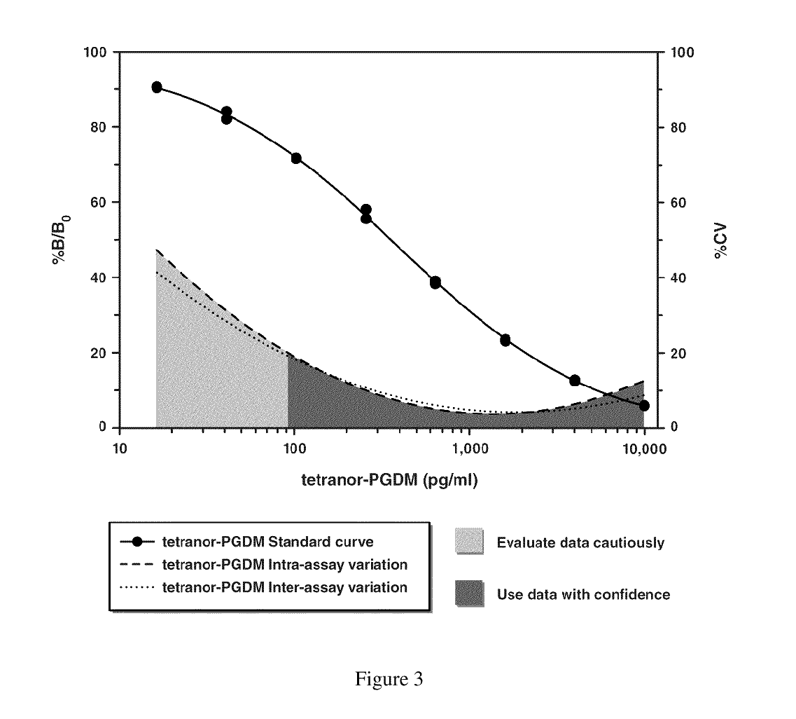

The standard curve shown in FIG. 3 is an example of the data typically produced using the procedure described herein. Results vary from assay run to assay run; therefore, a new standard curve must be run with each new assay performed.

### Step D2b: Precision

The intra- and interassay CV's have been determined at multiple points on the standard curve shown in FIG. 3. These data are tabulated below:

| Dose | % CV** | % CV** |
|---|---|---|
| (pg/mL) | Intraassay variation | Interassay variation |
| 10,000 | 10.6 | 5.4 |
| 4,000 | 6.9 | 6.5 |
| 1,600 | 6.3 | 8.4 |
| 640 | 7.7 | 7.7 |
| 256 | 8.5 | 9.5 |
| 102 | 17.0 | 16.7 |
| 41 | outside usable range | 20.8 |
| 16 | outside usable range | outside usable range |

**% CV represents the variation in concentration (not absorbance) as determined using a reference standard curve.

### Step D2c: Specificity of the Tetranor-PGDM EIA Antiserum

| Compound | Cross-reactivity (% XR) |
|---|---|
| tetranor-PGJM | 376% |
| tetranor-PGDM | 100% |
| tetranor-PGEM | 0.3% |
| tetranor-PGFM | <0.01% |
| Prostaglandin A₂ | <0.01% |
| Prostaglandin D₂ | <0.01% |
| 13,14-dihydro-15-keto Prostaglandin D₂ | <0.01% |
| Prostaglandin E2 | <0.01% |
| 13,14-dihydro-15-keto Prostaglandin E₂ | <0.01% |
| Prostaglandin F_{2α} | <0.01% |
| 13,14-dihydro-15-keto Prostaglandin F_{2α} | <0.01% |
| Prostaglandin J₂ | <0.01% |

It is understood for purposes of this disclosure that various changes and modifications may be made to the invention that are well within the scope of the invention. Numerous other changes may be made which will readily suggest themselves to those skilled in the art and which are encompassed in the spirit of the invention disclosed herein.

What is claimed is:

1. An immunogen comprising a tetranor-PGDM-carrier protein conjugate, a tetranor-PGJM-carrier protein conjugate, or a tetranor-PGJM-carrier protein Michael adduct.

2. The immunogen of claim 1, wherein the tetranor-PGDM-carrier protein conjugate, the tetranor-PGJM-carrier protein, or the tetranor-PGJM-carrier protein Michael adduct is formed by coupling a carrier protein to tetranor-PGDM or by coupling a carrier protein to tetranor-PGJM.

3. The immunogen of claim 2, wherein said carrier protein comprises keyhole limpet hemocyanin, bovine serum albumin, ovalbumin or thyroglobulin.

4. A method for forming an antibody that specifically binds to tetranor-prostaglandin D₂ metabolite (tetranor-PGDM) or tetranor-prostaglandin J₂ metabolite (tetranor-PGJM), the method comprising immunizing a biological species with the immunogen of claim 1.

5. The method of claim 4, wherein said biological species comprises a mammal.

6. The method of claim 5, wherein said mammal comprises a rabbit.

7. The method of claim 5, wherein said mammal comprises a mouse.

8. An antibody formed according to the method of claim 4, wherein said antibody comprises a monoclonal antibody or a polyclonal antibody.

9. An assay kit for measuring tetranor-PGDM metabolite levels or tetranor-PGJM metabolite levels in biological fluids comprising:
a tracer comprising tetranor-PGDM or tetranor-PGJM covalently bonded to a molecular tag; and
the antibody of claim 8.

10. The assay kit of claim 9, wherein said molecular tag comprises acetylcholinesterase, horseradish peroxidase, alkaline phosphatase, rhodamine, or fluorescein.

11. A method for forming an immunogen comprising a tetranor-PGDM-carrier protein conjugate or a tetranor-PGJM-carrier protein conjugate, the method comprising:
preparing a reaction mixture comprising tetranor-PGDM or tetranor-PGJM, an alkyl chloroformate, and a tertiary amine base;
forming a reaction mixture concentrate from said reaction mixture;
forming a second reaction mixture by adding a mixture comprising a carrier protein and a suitable aqueous buffer solution to said reaction mixture concentrate; and
forming a metabolite carrier-protein conjugate solution by purifying said second reaction mixture.

12. The method of claim 11, wherein said alkyl chloroformate comprises isobutyl chloroformate.

13. The method of claim 11, wherein said tertiary amine base comprises N,N-diisopropylethylamine.

14. The method of claim 11, wherein said carrier protein comprises keyhole limpet hemocyanin, bovine serum albumin, ovalbumin or thyroglobulin.

15. The method of claim 11, wherein the molar ratio of tetranor-PGDM or tetranor-PGJM to alkyl chloroformate and to tertiary amine base in said reaction mixture is from 1:0.1:0.2 to 1:2:5.

16. The method of claim 11, wherein the molar ratio of tetranor-PGDM or tetranor-PGJM to tertiary amine base and to alkyl chloroformate in said reaction mixture is 1:1:1.

17. The method of claim 11, wherein the molar ratio of tetranor-PGDM or tetranor-PGJM to tertiary amine base and to alkyl chlorofommate in said reaction mixture is 1:0.2:3.8

18. The method of claim 11, wherein the molar ratio of alkyl chloroformate with respect to tetranor-PGDM or tetranor-PGJM is from 10 to 200 mole percent.

19. The method of claim 11, wherein the molar ratio of alkyl chloroformate with respect to tetranor-PGDM or tetranor-PGJM is from 10 to 30 mole percent.

20. The method of claim 11, wherein the molar ratio of tertiary amine base with respect to tetranor-PGDM or tetranor-PGJM is from 20 to 500 mole percent.

21. The method of claim 11, wherein the molar ratio of tertiary amine base with respect to tetranor-PGDM or tetranor-PGJM is from 300 to 400 mole percent.

22. An immunogen formed according to the method of claim 11.

23. A method for forming an antibody that specifically binds to tetranor-prostaglandin D₂ metabolite (tetranor-PGDM) or tetranor-prostaglandin J₂ metabolite (tetranor-PGJM), the method comprising immunizing a biological species with the immunogen formed according to claim 11.

24. The method of claim 23, wherein said biological species comprises a mammal.

25. The method of claim 23, wherein said mammal comprises a rabbit.

26. The method of claim 23, wherein said mammal comprises a mouse.

27. An antibody formed according to the method of claim 23, wherein said antibody comprises a monoclonal antibody or a polyclonal antibody.

28. An assay kit for measuring tetranor-PGDM metabolite levels or tetranor-PGJM metabolite levels in biological fluids comprising:
a tracer comprising tetranor-PGDM or tetranor-PGJM covalently bonded to a molecular tag; and
the antibody of claim 27.

29. The assay kit of claim 28, wherein said molecular tag comprises acetylcholinesterase, horseradish peroxidase, alkaline phosphatase, rhodamine, or fluorescein.

30. A method for forming an immunogen comprising a tetranor-PGJM-carrier protein Michael adduct, the method comprising:
preparing a derivatized carrier protein mixture by contacting a carrier protein with N-succinimidyl-S-acetylthioacetate or N-succinimidyl-S-acetylthiopropionate;
purifying said derivatized carrier protein mixture;
forming a sulfhydryl-deprotected carrier protein mixture by treating said purified derivatized carrier protein mixture with hydroxylamine;
purifying said sulfhydryl-deprotected carrier protein mixture;
forming a tetranor-PGJM-carrier protein Michael adduct by treating said purified sulfhydryl-deprotected carrier protein mixture with tetranor-PGJM; and
purifying said tetranor-PGJM carrier protein Michael adduct.

31. The method of claim 30, wherein said carrier protein comprises bovine serum albumin, keyhole limpet hemocyanin, ovalbumin, or thyroglobulin.

32. A method for forming an antibody that specifically binds to tetranor-prostaglandin D₂ metabolite (tetranor-PGDM) or tetranor-prostaglandin J₂ metabolite (tetranor-PGJM), the method comprising immunizing a biological species with the immunogen formed according to claim 30.

33. The method of claim 32, wherein said biological species comprises a mammal.

34. The method of claim 32, wherein said mammal comprises a rabbit.

35. The method of claim 32, wherein said mammal comprises a mouse.

36. An antibody formed according to the method of claim 32, wherein said antibody comprises a monoclonal antibody or a polyclonal antibody.

37. An assay kit for measuring tetranor-PGDM metabolite levels or tetranor-PGJM metabolite levels in biological fluids comprising:
a tracer comprising tetranor-PGDM or tetranor-PGJM covalently bonded to a molecular tag; and
the antibody of claim 36.

38. The assay kit of claim 37, wherein said molecular tag comprises acetylcholinesterase, horseradish peroxidase, alkaline phosphatase, rhodamine, or fluorescein.

39. A method for forming a tracer comprising:
(a) forming a chemically-activated tetranor-PGDM or tetranor-PGJM species,
(b) chemically coupling said chemically-activated tetranor-PGDM or tetranor-PGJM species with an entity comprising a molecular tag to produce a substance comprising a tracer; and
(c) purifying said substance to produce a purified tracer.

40. A tracer formed in accordance with the method of claim 39.

41. A method for forming a tracer comprising:
(a) forming a mixture comprising tetranor-PGDM or tetranor-PGJM, a tertiary amine base, and a solvent;
(b) forming a reaction mixture by contacting the mixture with a second mixture comprising alkyl chloroformate and a solvent; and
(c) forming an activated tetranor-PGDM or tetranor-PGJM concentrate by removing said solvent;
(d) reconstituting said activated tetranor-PGDM or tetranor-PGJM concentrate in a solvent;
(e) producing a tracer mixture by chemically coupling said reconstituted activated tetranor-PGDM or tetranor-PGJM concentrate with an entity comprising a molecular tag; and
(f) purifying said tracer mixture.

42. A tracer formed in accordance with the method of claim 41.

43. A method for forming a tracer comprising:
(a) forming a mixture comprising tetranor-PGDM or tetranor-PGJM, N,N-diisopropylethylamine, and acetonitrile;
(b) forming a reaction mixture by contacting the mixture with a second mixture comprising alkyl chloroformate and acetonitrile; and
(c) forming an activated tetranor-PGDM or tetranor-PGJM concentrate by removing said acetonitrile;
(d) reconstituting said activated tetranor-PGDM or tetranor-PGJM concentrate in N,N-dimethylformamide;
(e) producing a tracer mixture by contacting said reconstituted activated tetranor-PGDM or tetranor-PGJM concentrate with acetylcholinesterase; and
(f) purifying said tracer mixture.

44. A tracer formed in accordance with the method of claim 43.

45. A method for forming a tracer comprising:
(a) forming a mixture comprising tetranor-PGJM and a suitable solvent;
(b) forming a second mixture comprising an entity comprising a molecular tag;
(c) combining said mixture with said second mixture to produce a reaction mixture; and
(d) purifying the reaction mixture.

46. A tracer formed in accordance with the method of claim 45.

47. A method for forming a tracer comprising:
(a) forming a mixture comprising tetranor-PGJM and N,N-dimethylformamide;
(b) forming a second mixture comprising acetylcholinesterase;
(c) combining said mixture with said second mixture to produce a reaction mixture; and
(d) purifying said reaction mixture.

48. A tracer formed in accordance with the method of claim 47.

49. A method for forming a tracer comprising a tetranor-PGJM-enzyme Michael adduct, the method comprising:
preparing a derivatized enzyme mixture by contacting an enzyme with N-succinimidyl-S-acetylthioacetate or N-succinimidyl-S-acetylthiopropionate;
purifying said derivatized enzyme mixture;
forming a sulfhydryl-deprotected enzyme mixture by treating said purified derivatized enzyme mixture with hydroxylamine;
purifying said sulfhydryl-deprotected enzyme mixture;
forming a tetranor-PGJM-enzyme Michael adduct by treating said purified sulfhydryl-deprotected enzyme mixture with tetranor-PGJM; and
purifying said tetranor-PGJM enzyme Michael adduct.

50. The method of claim 50, wherein said enzyme comprises acetylcholinesterase.

51. A tracer comprising:
a molecular tag; and
tetranor-PGDM or tetranor-PGJM covalently bonded to said molecular tag.

52. The tracer of claim 52, wherein said molecular tag comprises acetylcholinesterase, horseradish peroxidase, alkaline phosphatase, rhodamine, or fluorescein.

53. An assay kit for measuring tetranor-PGDM metabolite levels or tetranor-PGJM metabolite levels in biological fluids comprising:
a tracer comprising tetranor-PGDM or tetranor-PGJM covalently bonded to said molecular tag; and
an antibody that specifically binds to tetranor-prostaglandin D₂ metabolite (tetranor-PGDM) or tetranor-prostaglandin J₂ metabolite (tetranor-PGJM).
